(19) United States
(12) Patent Application Publication
(10) Pub. No.: US 2023/0355940 A1
(43) Pub. Date: Nov. 9, 2023

(54) OXYGENATING WOUND CARE DEVICE AND METHODS

(71) Applicant: Advance Vascular Inc., Little Rock, AR (US)

(72) Inventors: Chandan Das, Portland, OR (US); Trygve Burchardt, Howell, MI (US); Kundan Das, Little Rock, AR (US)

(21) Appl. No.: 18/144,089

(22) Filed: May 5, 2023

Related U.S. Application Data
(60) Provisional application No. 63/338,655, filed on May 5, 2022.

(51) Int. Cl. A61M 35/00

(52) U.S. Cl. CPC A61M 35/30; A61M 2202/0208

(57) ABSTRACT

Devices and methods for supplying oxygen to a patient for treatment of a wound or condition are provided. An outer housing includes a user contact surface with protrusions for penetrating biofilms of the wound and delivering oxygen produced by an onboard oxygen generating subsystem which electrochemically generates oxygen using an onboard power supply. The user contact surface and the protrusions are gas permeable to absorb and transmit generated oxygen into the wound to improve healing or treat the condition.

### CROSS-REFERENCE TO RELATED APPLICATIONS

This application claims the benefit of U.S. Provisional Application Ser. No. 63/338,655 filed May 5, 2022, the disclosures of which are hereby incorporated by reference as if fully restated herein.

### TECHNICAL FIELD

Exemplary embodiments relate generally to an oxygenating wound care device and systems and methods for manufacturing and/or operating the same or components thereof.

### BACKGROUND AND SUMMARY OF THE INVENTION

It is known to provide oxygen to certain kinds of wounds to improve healing. Certain oxygen rich creams or emulsions are known which may be applied to wounds, but these generally provide a short benefit and are difficult to control. Other known systems for providing oxygen to wounds, such as hyperbaric oxygen therapy, are often bulky and require equipment which prevents or limits patient mobility. These systems may also be inefficient in providing oxygen to the wound itself.

An oxygenating wound care device and related systems and methods are disclosed which provide oxygen to wounds in a manner which allows or increases patient mobility and/or which delivers oxygen in an efficient manner. An oxygen generating subsystem may be housed within an inner enclosure. The oxygen generating subsystem may comprise a nickel electrode and a combination oxygen/hydrogen electrode, such as with an aqueous electrolyte solution, gel, and/or saturated material between or about such components, though other materials may be utilized. Alternatively, a separate oxygen electrode and hydrogen electrode may be used. Voltage may be supplied in a controller manner to the nickel electrode to selectively oxidize Ni(OH2) to NiOOH to produce hydrogen and selectively reduce NiOOH to Ni(OH)2 to produce oxygen. Alternatively, the subsystem may be maintained in oxygen producing mode. In exemplary embodiments, the nickel electrode may be provided as a mesh which may be separated from the combination oxygen/hydrogen electrodes by one or more dielectrics, such as but not limited to a paper wick and/or one or more other wholly or partially non-conductive materials.

The inner enclosure may be gas permeable but liquid impermeable to seal the subsystem, such as against electrolyte leakage or other liquid intrusion, while permitting oxygen to escape into a larger enclosure. The larger enclosure may house certain components, such as a power supply, controller, sensors, indicators, and the like, at least some of which may be used to operate the oxygen generating subsystem.

The larger enclosure may include one or more porous layers. The porous layer(s) preferably face the wound and comprises one or more materials with high oxygen diffusivity, such as silicone, to permit diffusion of the produced oxygen through the layer and into the wound. Protrusions may extend from the porous layer to penetrate through biofilms of the wound and/or further disburse oxygen. In exemplary embodiments, without limitation, the protrusions may be sized to limit or prevent bending upon contact with a wound, particularly when penetrating biofilms. This may assist with penetration through one or more biofilms covering some or all of the wound and/or permit greater pressure offloading and oxygen delivery to the wound where it may be used for healing. Exemplary protrusions may be between 1 and 2 mm in diameter, by way of non-limiting example.

Alternatively, or additionally, the device may comprise an outer housing. The outer housing may include a user contact surface having the protrusions. A first portion of the outer housing may comprise one or more cavities for portions of a control subsystem (e.g., controller, battery, printed circuit board, etc.) and/or the oxygen generation subsystem (e.g., the electrolytes), respectively. Channels may extend between the cavities for an anode and cathode, respectively, to facilitate electrical transmission and oxygen generation. An internal cover may secure the control subsystem and keep the sensitive electronics separated from the electrolytes. Oxygen produced may be absorbed through, and diffuse through, the user contact surface and protrusions into the wound.

One or more debridement devices, such as an ultrasonic producing device, may be provided within the larger enclosure, such as to aid in wound cleaning and healing.

The integrated oxygen production may permit patient increased mobility. The device may be relatively compact and lightweight, thereby also permitting a high level of patient mobility.

The entire device may be disposable and/or relatively low cost, such as to permit regular removal and/or changing. Alternatively, or additionally, the device may be periodically recharged by operating the subsystem in a hydrogen producing mode and/or replacing electrolytes. In such embodiments, for example without limitation, the device may remain covering the wound over a long period of time, so as to promote healing.

The device may be particularly beneficial for low air circulation environments, such as but not limited to, wounds below normally clothed areas, within casts, braces, splints, or other device, combinations thereof, or the like. The device and/or subsystem may be integrated with traditional bandages or other wound coverings.

Further features and advantages of the systems and methods disclosed herein, as well as the structure and operation of various aspects of the present disclosure, are described in detail below with reference to the accompanying figures.

### BRIEF DESCRIPTION OF THE DRAWINGS

In addition to the features mentioned above, other aspects of the present invention will be readily apparent from the following descriptions of the drawings and exemplary embodiments, wherein like reference numerals across the several views refer to identical or equivalent features, and wherein:

FIG. 1 is simplified plan view of exemplary oxygen generation subsystem and process;

FIG. 2A is an exploded perspective view of an exemplary wound care device with the oxygen generation subsystem of FIG. 1;

FIG. 2B is a rear, partially exploded perspective view of the wound care device of FIG. 2A with an outer enclosure provided in exploded view and an inner enclosure provided in a collapsed view;

FIG. 3 is a side sectional view of another exemplary embodiment of the wound care device of FIGS. 2A-2B;

FIG. 4A is a perspective view of an exemplary manufacturing process underway for a component of the wound care device of FIGS. 2A-3;

FIG. 4B is a perspective view of another exemplary manufacturing process underway for a component of the wound care device of FIGS. 2A-3;

FIG. 5A is a side view of an exemplary controller for the wound care device of FIGS. 2A-3 and 9;

FIG. 5B is a top view of the electronics board of FIG. 5A;

FIG. 6A is a top view of an exemplary tissue interaction component of the wound care device of FIGS. 2A-3;

FIG. 6B is a side view of the tissue interaction component of FIG. 6A;

FIG. 7A is a top view of an exemplary upper component for the wound care device of FIGS. 2A-3 also illustrating section line A-A;

FIG. 7B is a side sectional view of the upper component of FIG. 7A taken along section line A-A;

FIG. 8 is an exploded perspective view of the exemplary wound care device of FIG. 2A with an exemplary debridement aid;

FIG. 9 is an exploded perspective view of another exemplary embodiment of the wound care device;

FIG. 10A is a perspective view of the wound care device of FIG. 9 assembled with an exemplary indicator not illuminated;

FIG. 10B is a perspective view of the wound care device of FIG. 10A with the indicator illuminated;

FIG. 11A is a front perspective view of an exemplary first layer of the wound care device of FIG. 9 shown in isolation;

FIG. 11B is a rear perspective view of the first layer of FIG. 11A;

FIG. 12A is a front perspective view of an exemplary second layer of the wound care device of FIG. 9 shown in isolation;

FIG. 12B is a rear perspective view of the second layer of FIG. 12A;

FIG. 13A is a front perspective view of a cover layer of the wound care device of FIG. 9 shown in isolation;

FIG. 13B is a rear perspective view of the cover layer of FIG. 13A;

FIG. 14A is a side view of an exemplary controller for the wound care device of FIGS. 2A-3 and 9;

FIG. 14B is a top view of the electronics board of FIG. 14A;

FIG. 15A is a side view of another exemplary controller for the wound care device of FIGS. 2A-3 and 9;

FIG. 15B is a top view of the electronics board of FIG. 15A;

FIG. 16 is a plan view of an exemplary wound care device provided at an exemplary wound;

FIG. 17 is a top perspective view of another exemplary embodiment of the wound care device;

FIG. 18 is a bottom perspective view of the wound care device of FIG. 17;

FIG. 19 is a top perspective view of an exemplary top housing for the wound care device of FIGS. 17-18;

FIG. 20 is a bottom perspective view of an exemplary internal cover for the wound care device of FIGS. 17-19;

FIG. 21 is a top perspective view of an exemplary internal cover for the wound care device of FIGS. 17-20;

FIG. 22 is an exploded view of the wound care device of FIGS. 17-21;

FIG. 23 is a top view of the first portion of the wound care device of FIG. 22 with certain exemplary dimensions noted;

FIG. 24A is a bottom view of an exemplary printed circuit board (PCB) of the wound care device of FIG. 22;

FIG. 24B is a top view of the PCB of FIG. 24A; and

FIG. 25 is a detailed side view of another exemplary embodiment of a projection at the user contact surface.

### DETAILED DESCRIPTION OF EXEMPLARY EMBODIMENT(S)

Various embodiments of the present invention will now be described in detail with reference to the accompanying drawings. In the following description, specific details such as detailed configuration and components are merely provided to assist the overall understanding of these embodiments of the present invention. Therefore, it should be apparent to those skilled in the art that various changes and modifications of the embodiments described herein can be made without departing from the scope and spirit of the present invention. In addition, descriptions of well-known functions and constructions are omitted for clarity and conciseness.

Embodiments of the invention are described herein with reference to illustrations of idealized embodiments (and intermediate structures) of the invention. As such, variations from the shapes of the illustrations as a result, for example, of manufacturing techniques and/or tolerances, are to be expected. Thus, embodiments of the invention should not be construed as limited to the particular shapes of regions illustrated herein but are to include deviations in shapes that result, for example, from manufacturing.

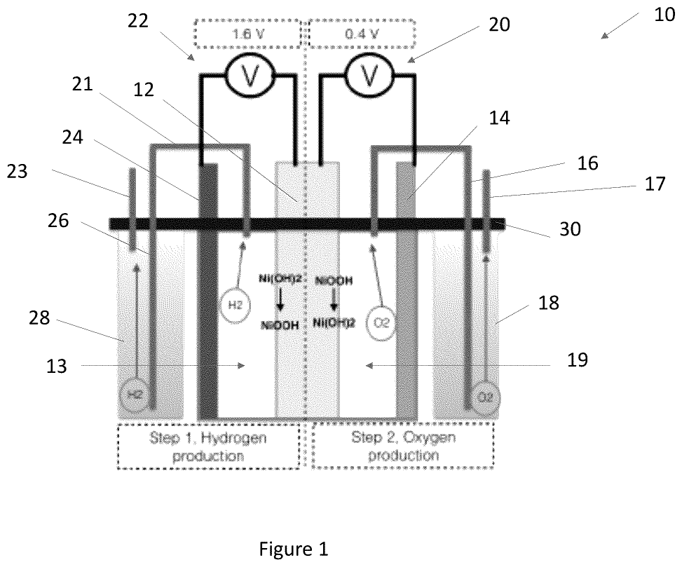

FIG. 1 illustrates an exemplary oxygen generation subsystem 10. Electricity may be provided from a first source 20 which is connected to a first electrode 12 and/or a second electrode 14. The first electrode 12 may comprise nickel. The second electrode 14 may comprise an oxygen electrode. Oxygen may be generated in a space 19 between the first and second electrodes 12 and 14, respectively. The space 19 may be fully or substantially sealed, such as by way of a barrier 30, the first electrode 12 and/or the second electrode 14. The space 19 may comprise electrolytes, such as in an aqueous solution, gel, and/or saturated material. The electrolytes may comprise sodium chloride (NaCL) and/or other ionic compounds in exemplary embodiments, without limitation. Alternatively, or additionally, the electrolytes may comprise potassium hydroxide (KOH) and/or other ionic compounds. The electrolytes may be provided as a fluid, gel, or absorbed into a material (e.g., cloth) filling some or all of the space 19. When in gel form, the electrolyte may comprise cross-linked polymer, such as but not limited to, poly acrylic acid.

A tube 16 may extend into the space 19 to capture released oxygen for transportation into a chamber 18. The chamber 18 may be fully or partially sealed, such as by way of the barrier 30 (or another barrier). The chamber 18 may be empty or comprise one or more other liquids or gasses. The chamber 18 may be configured to remove any trace electrolytes picked up with the oxygen from the space 19. A second tube 17 may extend into the chamber 18 to permit release of generated oxygen from the chamber 18. While tubes may be shown and/or described in certain embodiments, without limitation, other size, shape, kind, and/or types of passageways may be provided.

Electricity may be provided from a second power source 22 which is connected to the first electrode 12 and/or a third electrode 24. The third electrode 24 may comprise a hydrogen electrode. Oxygen may be generated in a space 13 between the first and third electrodes 12 and 24, respectively. The space 13 may be fully or substantially sealed, such as by way of the barrier 30 (or another barrier), the first electrode 12 and/or the third electrode 24. The space 13 may comprise electrolytes, such as in an aqueous solution, gel, and/or saturated material. The electrolytes may comprise sodium chloride and/or other ionic compounds in exemplary embodiments, without limitation. The electrolytes may be provided as a fluid, gel, or absorbed into a material (e.g., cloth) filling some or all of the space 13. When in gel form, the electrolyte may comprise cross-linked polymer, such as but not limited to, poly acrylic acid.

A third tube 21 may extend into the space 13 to capture released hydrogen for transportation into a second chamber 28. The second chamber 28 may be empty or comprise one or more gasses and/or liquids. The second chamber 28 may be fully or partially sealed, such as by way of the barrier 30 (or another barrier). The second chamber 28 may be configured to remove any trace electrolytes picked up with the hydrogen from the space 13. A fourth tube 23 may extend into the second chamber 28 to permit release of generated oxygen from the second chamber 28.

Hydrogen may be generated by connecting the second power source 22 to the first electrode 12 and the third electrode 24. Oxygen may be generated by connecting the first power source 20 to the first electrode 12 and the second electrode 14. Alternatively, or additionally, the first and/or second power sources 20, 22 may remain connected to the electrodes 12, 14, 24 and the power supply may be regulated (e.g., periodically turned on/off). Hydrogen and oxygen may be generated separately or simultaneously. In exemplary embodiments, without limitation, hydrogen is first produced and then oxygen. The process may be repeated.

The second power source 22 may be configured to provide about 1.5-1.6 volts, or higher, of electrical potential in exemplary embodiments, without limitation. This may be configured to cause Ni(OH)2 to oxidize to NiOOH. The first power source may be configured to provide about 0.2-0.4 volts, or higher, of electrical potential in exemplary embodiments, without limitation. This may be configured to cause NiOOH to be reduced to Ni(OH)2. In this manner, hydrogen and oxygen may alternately be produced using the common first electrode 12 depending on what power source 20 or 22 is applied.

In certain exemplary embodiments, without limitation, the first and second power sources 20 and 22 may be provided from a common source, such as but not limited to, one or more batteries connected to a potentiometer, switches, resistors, and/or other electrical components and/or control systems.

Electrical connection between the power source(s) 20, 22 and the various electrodes 12, 14, 24 may be selectively made, such as by attaching leads, or may be permanently established pathways which are controlled, such as by one or more switches or other devices.

In certain exemplary embodiments, without limitation, the second electrode 14 and the third electrode 24 may be provided as a combination electrode. One or more switches may be used to shift the polarity of the oxygen/hydrogen combination electrode 14, 24 and the first electrode 12, such as by controlling the amount of power supplied from the common or separate power sources 20, 22 to move between hydrogen and oxygen evolution modes of the subsystem 10.

The subsystem 10 may offer a number of benefits, such as but not necessarily limited to the following: the ability to create essentially or completely pure oxygen production without the need for complex membrane, separators or pressure control systems; relatively high oxygen evolution rates at relatively low voltages, which may permit a compact design with relatively high energy efficiency for oxygen evolution; use of alkaline electrolyte with proven long lifetime stability and high tolerance for impurities; a chemical (NiOOH) reservoir that enables oxygen evolution at relatively low voltages that is stable for long lifetime storage (years) and has low hazard, fire or explosion risk; ability to “charge” the Ni(OH)2 electrode under pure hydrogen production at energy levels below traditional water electrolysis methods for hydrogen production; scalability; use of materials that have been well tested for long lifetime durability in other applications; combinations thereof; or the like.

Ni(OH)2 to NiOOH may have a capacity of 210 mAh/g. At a rate of oxygen production of 10 L/min, for example, this is equal to a current of about 2.8 A or 4.76 g of Ni(OH)2/hour of oxygen production is required. This is just one example provided for illustration and without limitation.

In certain exemplary embodiments, without limitation, the subsystem 10 alternates between hydrogen production mode and the other in oxygen production mode. The modes may be subsequently flipped, such as within given intervals, to cover a continuous oxygen demand, for example. With only one system operating, the subsystem 10 may be scaled to fit the operational need over the required time period.

For a disposable oxygen wound dressing the amount of NiOOH in the first electrode 12 may be adjusted to match the time and oxygen production flow rate desired in exemplary embodiments, without limitation. For example, if a flow rate of 10 mL/h is desired for 165 hours and the capacity of the first electrode 12 is 150 mAh/g (a density of 2 g/cm3) the thickness of a 10 cm by 10 cm footprint first electrode 12 in the wound dressing may be provided at 2 mm or less. This is just one example provided for illustration and without limitation.

The first electrode 12, in exemplary embodiments without limitation, may be provided in the form of a powder, or a powder mixed with one or more binders to make a thin layer that is pasted onto a current collector, by way of non-limiting example. The current collector may be connected to one or more wires to lead the current out of a bag or other fully or partially sealed compartment, in exemplary embodiments.

By way of non-limiting example, within a 400 cm3 to 500 cm3 container oxygen evolution of 10 L/min to 20 L/min can be achieved with the subsystem 10. This may be sufficient to provide the oxygen needed in a number of other emergency situations, for example, i.e., for respiratory support, by only using a small handheld/portable oxygen generation subsystem 10. This is just one example provided for illustration and without limitation.

Other types and/or kinds of oxygen generation subsystems 10 may be utilized, such as in addition to, or alternative to, those shown and/or described with regard to FIG. 1. Such oxygen generation subsystems 10 may comprise any type or kind of oxygen generation and/or provision technology such as but not limited to, compressed oxygen, electrochemical technology, biochemical technology (e.g., hydrogen peroxide and vinegar), proton exchange membranes, pressure swing absorption, membrane separation, combinations thereof, or the like.

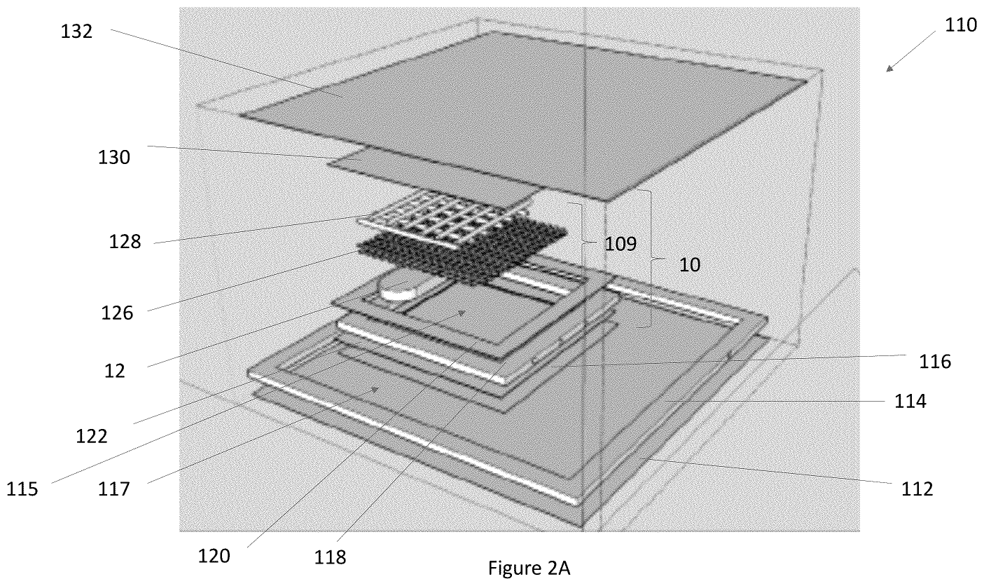

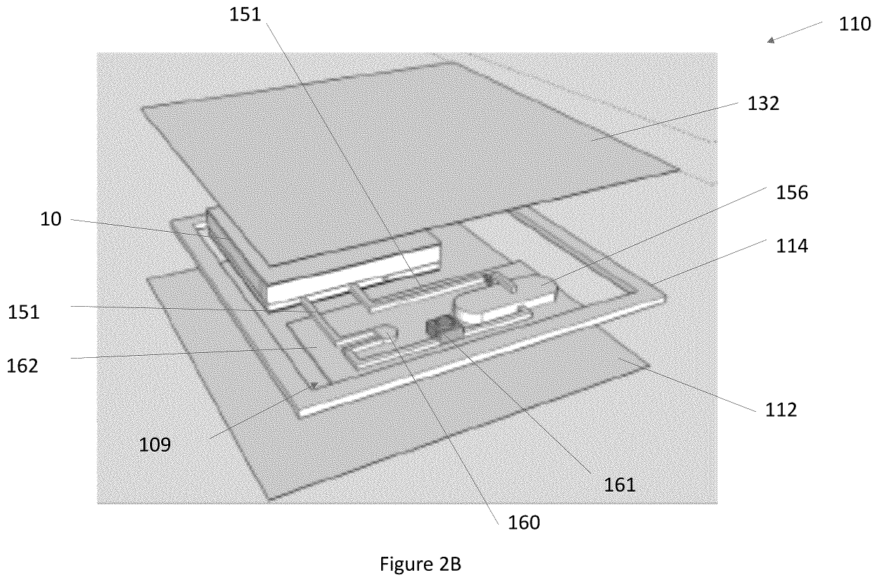

FIG. 2A and FIG. 2B illustrates an exemplary wound care device 110 with the oxygen generation subsystem 10. FIG. 2a is a fully exploded view, including an exploded view of some or all components of the subsystem 10. FIG. 2b is a partially exploded view, such as with some or all of the components of the subsystem 10, and/or an enclosure for the same, shown in a non-exploded view.

The device 110 may comprise the subsystem 10, though any type of oxygen generating device may be used. In exemplary embodiments, the device 110 may comprise a first layer or portion 112. The first portion 112 may be normally placed adjacent to a wound or other area of treatment. The first portion 112 may be comprise one or more gaseous impermeable or resistant materials, which may be configured to prevent the release of oxygen for example. The first portion 112 may be comprise one or more liquid impermeable or resistant layers or materials which may be configured to fully or substantially prevent liquid leakage. For example, without limitation, the first portion 112 may comprise one or more polymers. However, the first portion 112 may comprise one or more apertures, projections, filaments, needles, pores, combinations thereof, or the like, which permit oxygen or other gasses to flow, for example. Alternatively, or additionally, the first portion 112 may comprise one or more materials with a relatively high oxygen diffusivity, such as but not limited to silicone, to permit oxygen diffusion through the first portion 112. In this manner, the flow of oxygen may be directed to the wound, such as while trapping electrolytes, which may be in liquid or gel form.

The device 110 may comprise a second layer or portion 132. The second portion 132 may comprise one or more gaseous impermeable or resistant materials, which may be configured to fully or substantially prevent the release of oxygen or other gasses, for example. The second portion 132 may comprise one or more liquid impermeable or resistant layers or materials which may be configured to prevent liquid leakage. The second portion 132 may comprise one or more polymers.

The first and second portions 112 and 132 may comprise the same or different materials. A seal 114 may be provided between the first and second portions 112 and 132 to define an outer enclosure and/or a fully or partially sealed space 117. The outer enclosure may sometimes be referred to as an outer bag. The first portion 112, the second layer 132, and the seal 114 may define an enclosure for the space 117 in exemplary embodiments. The space 117 may be gas impermeable or resistant in exemplary embodiments, without limitation. In other exemplary embodiments, the space 117 may be normally, fully or substantially, gas-tight but may permit gas to exit through portions of the first portion 112 and/or by diffusion through some or all of the first portion 112. The space 117 may be liquid impermeable or resistant in exemplary embodiments, such as to trap the electrolytes in exemplary embodiments, without limitation. The seal 114 may normally be, fully or substantially, gas and/or liquid tight or resistant. The seal 114 may comprise one or more ports, such as for wires, needles, tubes, combinations thereof, or the like. For example, without limitation, the ports may permit filling the space 117 with electrolytes for replacing or otherwise, removing generated oxygen or other gasses, entry and exit of wires for providing power to the subsystem 10 or components thereof, combinations thereof, or the like. The seal 114 may comprise rubber in exemplary embodiments. The seal 114 may be 0.01-5 mm thick in exemplary embodiments, without limitation.

The device 110 may comprise a first inner layer or portion 116. The first inner layer 116 may comprise a plurality of porous of any size or shape configured to permit gas penetration but prevent liquid penetration in exemplary embodiments. The device 110 may comprise a second inner layer or portion 130. The second inner layer 130 may comprise a plurality of porous configured to permit gas penetration but prevent liquid penetration in exemplary embodiments. The first and/or second inner layers or portions 116 and 130 may comprise one or more polymers and may be the same or different materials, though such is not required. One or more inner seals 118, 120, 122 may be provided between the first and second layers or portions 116 and 130.

The first inner layer 116, the second inner layer 130, and/or the one or more inner seals 118, 120, 122 may define an inner enclosure and/or an inner space 115. The inner enclosure may sometimes be referred to as an inner bag. The inner space 115 may be fully or substantially gas permeable but liquid impermeable or resistant in exemplary embodiments. In this manner, generated gasses, such as oxygen, may be removed from the inner space 115 but electrolytes may be trapped, by way of non-limiting example. The inner space 115 may be configured to house some or all components of the subsystem 10 in exemplary embodiments.

A first one of the inner seals 118 may comprise rubber. A second one of the inner seals 120 may comprise a plastic film. A third one of the inner seals 122 may comprise a plastic film. The use of films may permit hot stamping to join certain various components of the device 110. The films may be used in conjunction with one or more adhesives, in exemplary embodiments, such as to permit sealing with silicon-based materials. Slight griding of the inner seals 118, 120, 122 may be performed to increase adhesion.

The one or more inner seals 118, 120, 122 may comprise one or more ports configured to accept liquids (e.g., electrolytes for recharging/replacement), gasses, accommodate tubes (e.g., 21, 23, 16, and/or 17) or other passageways of the same, and/or electrical wires 151 for passing therethrough while preventing some or all liquid from passing therethrough. One or more of the ports may be configured to permit oxygen or other gasses to escape, such as while preventing liquids from entering or escaping by way of valves, layers, combinations thereof, or the like. The inner seals 118, 120, 122 may be 0.01-5 mm thick in exemplary embodiments, without limitation.

The first electrode 12 may be positioned within the inner space 115. A combination electrode 128 may be provided within the inner space 115. The combination electrode 128 may comprise the second electrode 14 and the third electrode 24 of the subsystem 10 in exemplary embodiments, though separate electrodes may alternatively be utilized. The combination electrode 128 may be provided as a grid or mesh. The combination electrode 128 may comprise one or more metals, such as but not necessarily limited to, stainless steel, nickel, or the like. The combination electrode 128 may be stable in electrolyte. The combination electrode 128 may be coated with a catalyst in exemplary embodiments.

A separation layer 126 may extend between the first electrode and the combination oxygen/hydrogen electrode 128. The separation layer 126 may comprise one or more nonconductive materials, such as provided in a grid or mesh, particularly which are stable when exposed to an electrolyte. The separation layer 126 may comprise one or more dielectrics, such as but not limited to a paper wick and/or one or more other wholly or partially non-conductive materials. Where more than one electrode is used in lieu of the combination electrode 128, multiple separation layers 126 may be utilized. The separation layer 126 may comprise one or more materials having high ionic mobility to prevent ohmic resistance, and subsequent heating. Example of such materials include, but are not necessarily limited to, porous hydrophilic plastic films. A thickness of 0.01-4 mm may be utilized in exemplary embodiments. The separation layer 126 may be surrounded by, or filled with, electrolyte.

The first inner layer 116, the second inner layer 130, the one or more inner seals 118, 120, the first electrode 12, the separation layer 126, and/or the therethrough while preventing some or all liquid leaks. The first inner layer 116, the second inner layer 130, and/or the one or more inner seals 118, 120 may define an inner space 115. The inner space 115 may be gas permeable but liquid impermeable or resistant in exemplary embodiments. In this manner, oxygen or other gasses generated by the subsystem 10, or components thereof, may be released from the inner space 115 while trapping electrolytes in exemplary embodiments.

The subsystem 10 within the device 110 may comprise one or more of the first inner layer 116, the second inner layer 130, the one or more inner seals 118, 120, the first electrode 12, and/or the combination electrode 128.

The wiring 151, tubes (e.g., 21, 23, 16, and/or 17) or other passageways may pass into and/or out of the inner space 115 to provide electronic signals to, provide electrical power to, and/or supply or receive gasses (e.g., oxygen and/or hydrogen) from the subsystem 10 or components thereof within the inner space 115. However, in exemplary embodiments, the layers (e.g., 130, 116) of the inner space 115 may be configure to permit produced gas to escape the inner space 115 and circulate within the larger space 117. The wiring 151 may be connected to various components by welding, soldering, or the like. The wiring 151 may be configured to be stable and not corroded in the electrolyte.

A common power source 156 may be electrically connected to the subsystem 10 or components thereof. The common power source 156 may comprise, or act as, the first power source 20 and the second power source 22. The common power source 156 may comprise one or more batteries. Such batteries may be alkaline, lithium ion, and/or zinc/air type batteries, though any type and/or kind of battery may be utilized. Resistors may be provided to control voltage sourced from such batteries. Zinc/air type batteries, in particular, may be used as the current delivered may be proportional to the partial pressure of oxygen. Therefore, leaving the zinc/air type battery at a diffusion limited voltages for a short time (e.g., 1-20 seconds) a read of the oxygen concentration inside the device 110 may be achieved, reported, and/or tuned for optimal operation and/or treatment. In exemplary embodiments, the common power source 156 is connected to the first electrode 12 and the common electrode 128 to create an electrical potential for the generation of oxygen, hydrogen, combinations thereof, or other gasses.

An indicator 160 may be provided. The indicator 160 may be located within the larger space 117, but outside the inner space 115 in exemplary embodiments. In exemplary embodiments, without limitation, the indicator 160 is electrically interposed between the common power source 156 and the subsystem 10, or components thereof. The indicator 160 may comprise, or be connected to, one or more sensors, such as oxygen sensors, hydrogen sensors, and/or gas sensors. The indicator 160 may comprise, or be connected to, one or more lights, such as light emitting diodes. The indicator 160 may be configured to illuminate, deactivate, change color, flash, combinations thereof, or the like to indicate the flow, amount, lack of flow, presence, non-presence, concentration, combination thereof, or the like of certain gasses, such as oxygen, hydrogen, and/or other gasses.

One or more controllers 109 may be provided. The controller(s) 109 may be provided within the larger space 117 and outside of the inner space 115 in exemplary embodiments. The controller(s) 109 may comprise, and/or be configured to operate, the common power source 156, the indicator 160, combinations hereof, or the like. The controller(s) 109 may comprise, or be connected to, one or more resistors for controlling voltage provided, and thus if/when, how much, and/or what type of gasses are produced by the subsystem 10. Such resistor(s) may be fixed or variable.

An electronics chassis 162 may be provided for the power source 156, the indicator 160, wiring 151, combinations thereof, or the like. The electronics chassis 162 may comprise a plate, foil, PCB board, combinations thereof, or the like. Some or all of the indicator 160 may be exposed, and/or placed below transparent or translucent material so that it is viewable. The electronics chassis 162 may be provided within the larger space 117 and outside of the inner space 115 in exemplary embodiments. The electronics chassis 162 may be part of the controller 109.

In exemplary embodiments, without limitation, the common power source 156 may form part of, and/or be controlled by, the controller(s) 109, to provide a voltage sufficient to generate oxygen over at least a period of time without necessarily producing hydrogen. In this manner, by way of non-limiting example, oxygen may be produced for a desired amount of time. The device 110 may be later removed and placed in hydrogen producing mode to recharge the first electrode 12, such as for reuse, though such is not required.

The controller(s) 109 may comprise, or be connected to, one or more user input devices 161, such as but not limited to buttons, switches, touch pads or touch sensitive areas, relays, microphones (for audio commands), combinations thereof, or the like. Actuation of the user input device(s) 161 may be configured to activate, deactivate, and/or control functionality of the device 110 or components thereof, such as but not limited to the oxygen producing subsystem 10.

The device 110, in exemplary embodiments without limitation, may comprise a footprint of between 0.5 inches and 6 inches in a width dimension and between 0.5 inches and 6 inches in a length dimension. The device 110 may comprise a depth of between 0.1 inches and 2 inches, by way of non-limiting example. The size of the device 110, including onboard oxygen producing subsystem 10 may facilitate patient mobility. The device 110, including components thereof, may be relatively flexible, so as to permit conformity to a wound and/or patient mobility. The device 110 may be integrated with a larger bandage or securing device, though such is not required.

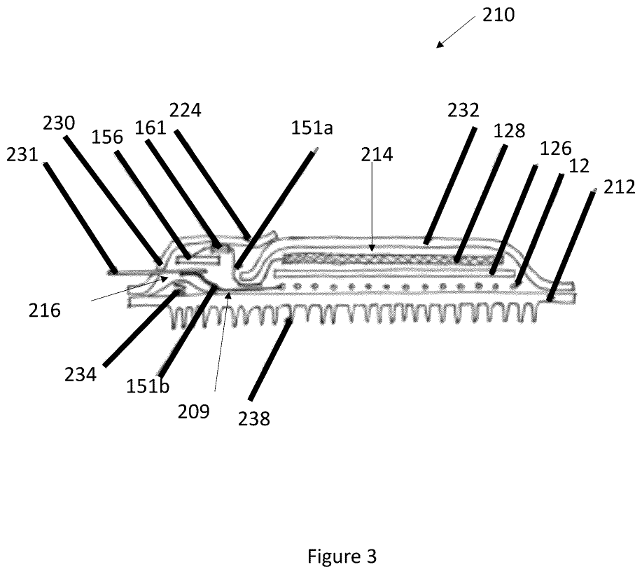

FIG. 3 illustrates another exemplary wound care device 210 with the oxygen producing subsystem 10. Similarly numbered components of the device 210 may be the same or similar to components of the device 110, though such is not necessarily the case.

A first layer 212 may comprise one or more protrusions 238. The protrusions 238 may be configured to penetrate biofilms, which sometimes form over a wound bed, especially in the case of chronic wounds. The protrusions 238 may be configured to provide deeper, penetrative oxygen delivery in a controlled manner. The first layer 212 may be configured to physically touch, or extend adjacent to, the wound. The first layer 212 may comprise one or more materials with a high oxygen affinity, such as but not limited to silicone, to permit oxygen absorption and transportation by diffusion. In exemplary embodiments, the protrusions 238 comprise fingerlike projections, and may be provided randomly or in a pattern at some or all of an underside of the first layer 212. Alternatively, or additionally, the first layer 212, including but not limited to the protrusions 238, may comprise one or more apertures, such as but not limited to micro- or nano-pores, to facilitate oxygen flow. The apertures may be provided randomly or in a pattern at some or all of the first layer 212, such as but not limited to the protrusions 238. The apertures may be sized to permit gas permeation of certain particles, such as but not necessarily limited to oxygen, while preventing permeation of liquids, such as water, blood, plasma, and the like. The apertures may be configured to permit one-way permeation, though such is not required. The first layer 212 may be configured to direct the flow of oxygen towards the wound, such as while trapping electrolytes.

The first electrode 12 may be provided above the first layer 212 in exemplary embodiments. The separation layer 126 may be provided above the first electrode 12 and below the combination electrode 128 in exemplary embodiments. The combination electrode 128 may comprise nickel, such as to prevent hydrogen formation. A second layer 232 may extend from one or more portions of the first layer 212 to define a first space 214 for certain components of the device 210. In exemplary embodiments, the second layer 232 may sandwich the first electrode 12, the separation layer 126, and the combination electrode 128 between the first layer 212 and the second layer 232 within the first space 214. In exemplary embodiments, without limitation, the second layer 232 may form a dome shape to accommodate these components.

A third layer 224 may extend from another portion of the first layer 212 to the second layer 232, in exemplary embodiments, to provide a second space 216. The second space 216 may accommodate certain other components of the device 210. Electrical wiring 151 may extend between components of the first space 214 and the second space 216 in exemplary embodiments. For example, without limitation, one or more wires 151 may extend between the first layer 212 and the second layer 232. In exemplary embodiments, without limitation, a first wiring pathway 151a may extend from the combination electrode 128 to the controller 209, and/or from the controller 209 to the common power source 156. In exemplary embodiments, without limitation, a second wiring pathway 151b may extend from the first electrode 12 to a tab 231.

The tab 231 may extend through an opening 230 in the third layer 224 to temporarily separate a portion of the wiring 151, such as but not limited to the terminal end of the second wiring pathway 151b, from the common power source 156. The tab 231 may comprise non-conductive material. The tab 231 may be configured to prevent premature activation of the subsystem 10. When a user is ready to use the device 210 and/or subsystem 10, the user may manually remove the tab 231, such as by pulling, to allow the wiring 151, such as but not limited to the terminal end of the second wiring pathway 151b, to contact the common power source 156 to establish an electrical circuit which may begin the production of oxygen.

One or more springs 234, such as but not limited to coil springs, may be provided within the second space 216. The spring(s) 234 may be configured to bias the wiring 151, such as the terminal end of the second wiring pathway 151b, against the tab 231 and/or common power source 156. Any number, type, kind, and/or arrangement of springs 234 may be utilized. In this manner, the second space 216, and any components therein, may be accessible while maintain relative seal of the first space 214.

The second layer 232 and/or the third layer 234 in exemplary embodiments may comprise one or more non-gaseous (e.g., oxygen) absorbing materials, such as to direct oxygen towards the wound adjacent the first layer 212, though such is not required.

Once wires 151 are connected to the first electrode 12 and/or the combination electrode 128, sheets or layers comprising silicone may be positioned on either or both sides of the first electrode 12, the combination electrode 128, and/or the separation layer 126 such as to fully or partially encase such components in silicone. Heat may be applied to bond the layers to the underlying components 12, 128, 126 and/or to each other.

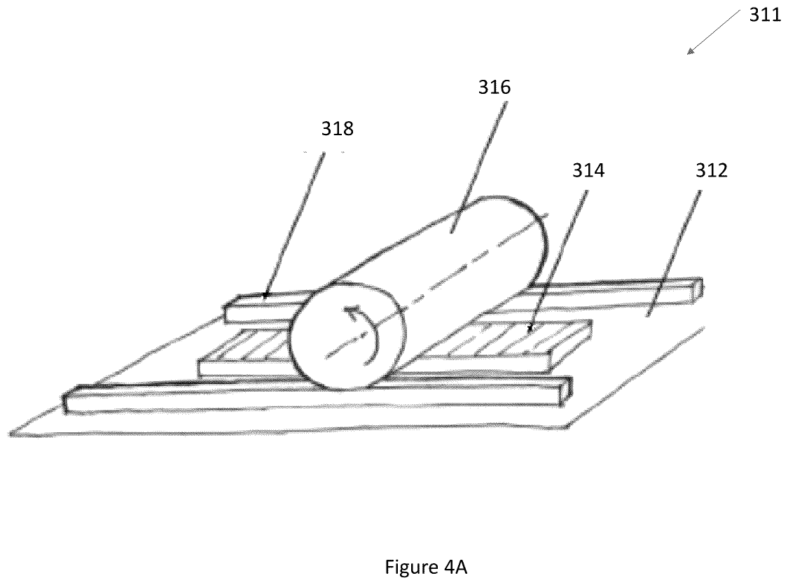

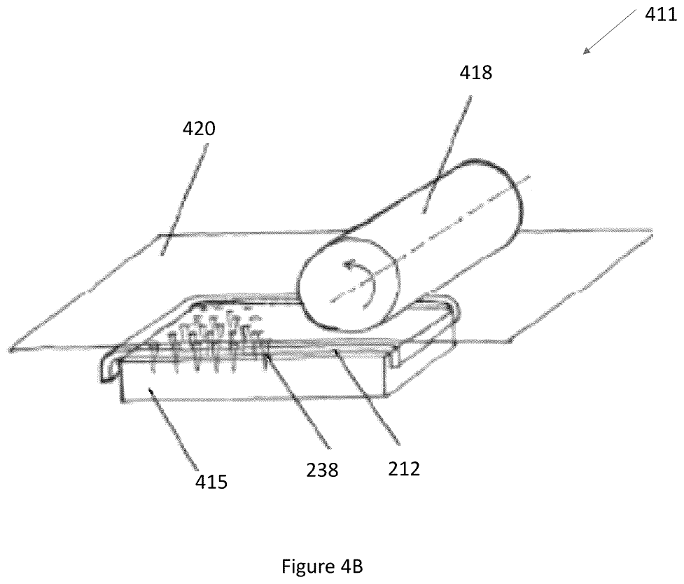

FIG. 4A illustrates an exemplary lamination/calendaring process 311 for manufacturing certain components of the device 210. The components may include, for example without limitation, the first portion 112, 212, the second layer 132, 232, the first inner layer 116, the second inner layer 130, and/or the third layer 230. In exemplary embodiments, without limitation, the process 311 is used to laminate and/or calendar silicon to form a material for enclosing the subsystem 10 within a soft, flexible wound dressing package. In exemplary embodiments, without limitation, the process 311 is used to create a layer which may be divided for use as the second layer 232 and/or the third layer 230.

A first sublayer 312 may be provided. The first sublayer 312 may comprise a non-stick material, such as but not limited to polytetrafluoroethylene (hereinafter also “PTFE”). A second sublayer 314 may be provided. The second sublayer 314 may comprise a liquid or paste comprising silicone, which may be provided in multiple parts that are mixed. The second sublayer 314 may comprise C6-750 liquid silicone rubber available from Dow Silicones Corp. of Midland MI (www.dow.com/en-us) in exemplary embodiments. Any type of kind of material may be used for the second sublayer 314. The second sublayer 314 may be placed atop to the first sublayer 312 and within a plurality of spacers 318. In exemplary embodiments, without limitation, two spacers 318 are provided along opposing edges of the first sublayers 312 to form rails. A calendar 316 may be coated with one or more materials, such as but not limited to one or more non-stick materials, such as but not limited to PTFE. The calendar 316 may be rolled across the spacers 318 to deposit the one or more materials to the second sublayer 314 and/or adhere the second sublayer 314 to the first sublayer 312. This arrangement may provide for consistent thickness of the resulting product and/or the various sublayers thereof.

The resulting product may be vacuum treated to remove trapped air or other bubbles. The resulting product may be heated, such as by placing in an over at 100-250° C. for a period of time, such as but not limited to, 1-3 hours to cure. Alternatively, or additionally, the resulting product may be cured by ultraviolet (UV) light exposure.

FIG. 4B illustrates another exemplary lamination/calendaring process 411 for manufacturing certain components of the device 210. The components may include, for example without limitation, the first portion 112, 212, the second layer 132, 232, the first inner layer 116, the second inner layer 130, and/or the third layer 230. In exemplary embodiments, without limitation, the process 311 is used to laminate and/or calendar silicon onto a perforated PTFE plate to form micro-textured surfaces that allows for oxygen distribution. In exemplary embodiments, without limitation, the process 311 is used to form the first layer 212.

A block 415 may be provided. The block 415 may comprise PTFE in exemplary embodiments. The block 415 may be perforated by drilling, etching, cutting, punching, combinations or the like to form a pattern of holes and/or cavities. A first layer 212 may be provided. The holes and/or cavities in the block 415 may be configured to create the protrusions 238. Material 416 for creating the first layer 212 may be deposited atop the block 415, such as in a liquid/paste form. The material 416 may be provided in multiple parts which may be mixed prior to application. A separating layer 420 may be provided atop the material 416. The material 416 may comprise silicon, which may be provided in multiple parts which are mixed. The material 416 may comprise C6-750 liquid silicone rubber available from Dow Silicones Corp. of Midland MI (www.dow.com/en-us) in exemplary embodiments. Any type of kind of material may be used for the material 416. The separating layer 420 may comprise one or more non-stick materials, such as but not limited to PTFE. A calendar 418 may be coated with one or more materials, such as but not limited to one or more non-stick materials, such as but not limited to PTFE. The calendar 418 may be rolled across the separating layer 420 to inject the material 416 into the block 415 to create the protrusions 238 and/or remainder of the first layer 212. Channels may alternatively or additionally cut into the block 415.

Alternatively, or additionally, silicon used may comprise MDX4-4210 from DuPoint, though any type or kind of silicone or material may be used.

The block 415 may be secured by a vacuum to a worktable. Some or all of the holes in the block 415 may be cut all the way through so as to pull the material 416 into the block 415 during the process 411 to provide for filling of very fine micro filaments. A tool with pins corresponding to the holes may be used to release the first layer 212 upon completion. This may permit removal without tearing.

The resulting product may be vacuum treated to remove trapped air or other bubbles. The resulting product may be heated, such as by placing in an over at 100-250° C. for a period of time, such as but not limited to, 1-3 hours to cure.

Alternatively, or additionally, injection molding may be used to create some or all components of the device 210, such as with or without micro texturing and/or microfilaments.

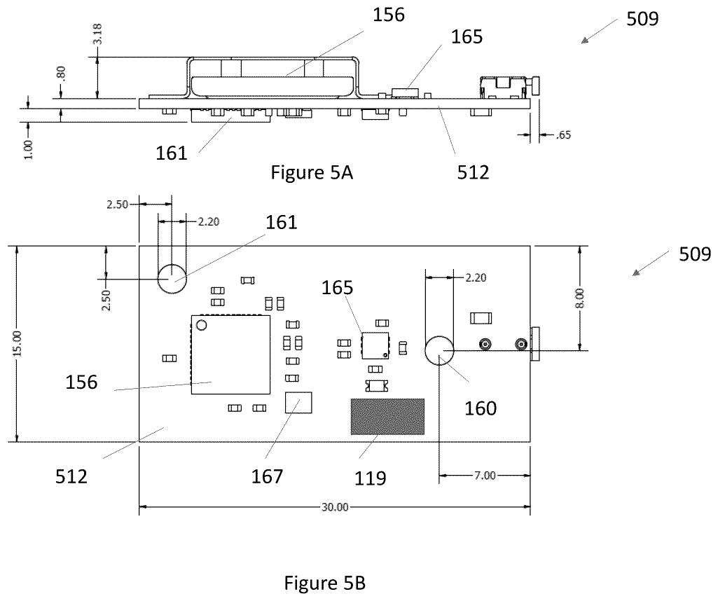

FIG. 5A and FIG. 5B illustrate an exemplary detailed view of a controller 509 for the device 210. The controller 509 may comprise a printed circuit board 512 (hereinafter also “PCB”). The PCB 512 may comprise embedded wiring, which may form some or all of the wiring 151. The PCB 512 may serve as the electronics chassis 162. The controller 509 may comprise the indicator 160, the user input device 161, the common power source 156, combinations thereof, or the like. The user input device 161 may be mounted to the PCB 512 in exemplary embodiments. The user input device 161 may serve as an alternative to, or in addition to, the tab 231 for activating the subsystem 10 and/or device 210. The user input device 161 may be part of, or connected with, the controller 509 in exemplary embodiments, without limitation. The user input device 161 may be depressible though the device 110, 210, 610 in exemplary embodiments or may comprise a portion which extends outside of the second and/or third layer 232, 224 in exemplary embodiments. One or more switches, dials, selectors, potentiometers, resistors, electrical components, electrical pathways, or the like may be provided to regulate the amount of oxygen produced or released by the device 210. Alternatively, or additionally, the controller 509 may be configured to accept electronic instructions in this regard. For example, without limitation, data related to a polarization curve may be stored at the controller 509 or elsewhere to determine an amount of oxygen produced as a function of voltage. Current or voltage may be measured to control an amount of oxygen produced. Predetermined oxygen settings may be provided, such as but no limited to, on, off, high, medium, low.

The PCB 512 may comprise one or more electrical regulating components 165. The electrical regulating components 165 may comprises resistor, capacitors, switches, diodes, inductors, gates, combinations thereof, or the like. Some or all of the electrical regulating components 165 may be electrically interposed between the common power source 156 and one or more of the various electrodes, such as but not limited to the first electrode 12, to control characteristics of the power supplied to the one or more of the various electrodes, such as but not limited to the first electrode 12. For example, without limitation, some or all of the electrical regulating components 165 may be configured to regulate voltage supplied to the first electrode 12 to move it between oxygen and hydrogen producing modes. More specifically, without limitation, the controller 509 may be configured to selectively route electricity from the common power source 156 through some or all of, or none or, the electrical regulating components 165 to provide a first voltage for generating hydrogen and at least some different ones, or none of, the electrical regulating components 165 to provide a second voltage for generating oxygen. Alternatively, or additionally, the controller 509 may be configured to alternate between an oxygen and hydrogen producing modes by reversing the direction of current flow. The controller 509 may be configured to alternate between oxygen and hydrogen producing modes based on time, time of use, amount or rate of oxygen produced, amount or rate of oxygen detected, ambient temperatures, ambient pressures, combinations thereof, or the like.

The PCB 512 may comprise one or more sensors 169. The sensors 169 may include, but are not necessarily limited to, gas sensors (e.g., oxygen sensors), power characteristics sensors (e.g., voltage sensors), temperature sensors, humidity sensors, motion sensors (e.g., gyroscope, accelerometer), pressure sensors, combinations thereof, or the like. Humidity, for example, may be monitored to determine if sufficient moisture is available to the wound for optimal healing. Pressure, for example, may be monitored to determine if adequate pressure is placed on the wound for optimal healing. Temperature, for example, may be monitored to determine if adequate circulation is provided (generally, lower temperatures might indicate less circulation) and/or if inflammation is being experienced (generally, higher temperatures indicate inflammation and possibly infection). Motion, for example, may be monitored to determine if too much or too little motion is being experienced for optimal healing. This information may be reported to the medical provider for providing treatment instructions or advice to the patient, for example. This information may be displayed local at the device 110 and/or at one or more remote devices. Alternatively, or additionally, the controller 509 may be configured to automatically generate alerts or notifications in this regard to devices associated with a healthcare provider, caretaker, patient, combinations thereof, or the like, such as where parameters exceed predetermined criteria. For example, without limitation, an electronic notification may be generated and transmitted, such as to the user of the device 110, 210, 610, a healthcare provider, and/or a third-party where movement levels above a pre-determined threshold are detected. As another example, without limitation, data regarding produced oxygen levels may be transmitted, such as to the user of the device 110, 210, 610, a healthcare provider, and/or a third-party. Some or all of the sensors 169, in exemplary embodiments without limitation, may be distributed through the device 110, 210, 610.

The PCB 512 may comprise one or more network communication devices 167. The network communication devices 167 may permit wired and/or wireless electronic communication with one or more external, remote devices, such as but not limited to smartphones, smart watches, tablets, personal computers, combinations thereof, or the like. Such communication may be achieved by way of wireless internet connectivity, near field communication (e.g., Bluetooth®), hardwired connection (e.g., ethernet), combinations thereof, or the like. Data communicated may include, for example without limitation, data from some or all of the sensors 169, certain status information (e.g., on/off state, amount of oxygen produced, first electrode 12 status, runtime, gyroscopic position information, pressure readings, temperature readings, etc.), notifications, warning, instructions, combinations thereof, or the like. Data may be stored and periodically transmitted. Any type of kind of data from any type of kind of sensor may be transmitted, including but not necessarily limited to, those shown and/or described herein. The data may aid in treatment decisions. Electronic control instructions may, alternatively or additionally, be sent to the device 210 using the network communication devices 167.

The controller 509 may comprise, or be connected to, one or more debridement aids 119. The debridement aids 119 may comprise ultrasonic devices, vibration generating components (e.g., motors, weights), a lightly abrasive material (e.g., microfiber cloth), gel (e.g., hydrogel), one or more chemicals (e.g., synthetic enzyme, clostridium, histolyticum, collagenase, varidase, papain, and bromelain), saline or other aqueous solution, combinations thereof, or the like. Some or all of the aforementioned components may be embedded in a cloth or other sterile material in exemplary embodiments. The debridement aids 119 may be periodically and/or occasionally activated, such as but not limited to by way of the controller 509 or separate controller, to mechanically agitate would tissues, such as to periodically clean the wound to promote healing, such as but not limited to for burn wounds. The debridement aids 119 may have an independent power supply, or utilize the common power supply 156.

In exemplary embodiments, without limitation, the controller 509 may be configured for automatic and/or manual device 210 operational adjustment. Such adjustment may be provided based on readings from the various sensors of the device 210. For example, without limitation, the readings may be remotely reported, reviewed by a healthcare provider, and operational instructions may be remotely relayed to the controller 509 to adjust operations of the device 210 (e.g., amount, rate, and/or time of oxygen production, level, time, or the like of operation of debridement aids 119, combinations thereof, or the like). Alternatively, or additionally, the readings may be analyzed by one or more algorithms configured to automatically adjust operations of the device 210 based on the readings accordingly. In this fashion, by way of non-limiting example, additional oxygen may be provided in the early stages of wound care and limited as healing progresses. If insufficient healing is experienced, additional oxygen may be provided, by way of non-limiting example.

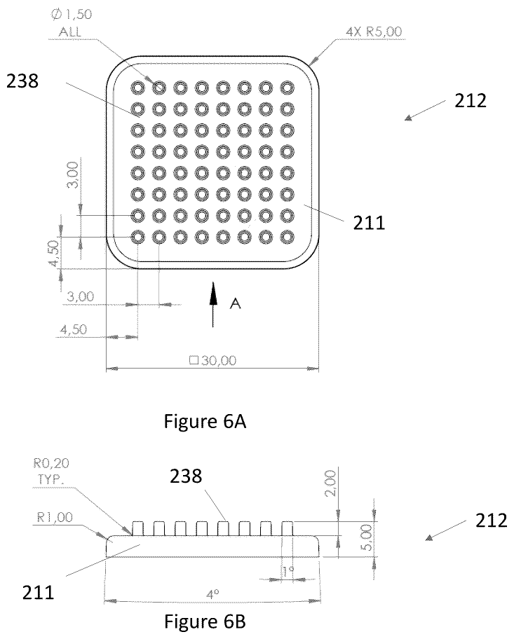

FIG. 6A and FIG. 6B illustrate another exemplary embodiment of the first layer 212. The projections 238 may extend from a base portion 211. The size, shape, number, and/or arrangement of the projections 238 is merely exemplary and is not intended to be limiting. In exemplary embodiments, without limitation, each of the projections 238 are at least 500 microns in diameter at their narrowest point. More preferably, each of the projections 238 are at least 1 mm in diameter at their narrowest point. For example, without limitation, each of the projections 238 may be substantially, or exactly, 1.5 mm in diameter at their widest point, though such is not required. Diameters between 1 mm and 5 mm may be utilized in exemplary embodiments, without limitation. Some or all of the projections 238 may vary in size and/or shape.

In other exemplary embodiments, without limitation, each of the projections 238 are 0.01-10 mm long and 0.01 to 3 mm thick. The projections 238 may comprise filament and/or needle like shapes. Various channels may be textured into the first layer 212, such as to the various projections 238 and/or in the wound facing portion of the first layer 212 for drainage and/or exudation from the wound. Such channels may be between 0.01 and 10 mm in height and width.

The projections 238 may be sized to prevent or limit bending. This may assist with breaking some or all of the projections 238 through biofilm(s) covering a wound which may assist with oxygen delivery, including better pressure offloading into the wound where oxygen may be utilized for healing. One example, without limitation, of such an advantageously sized projection 238 are 1.5 mm in diameter or thereabouts.

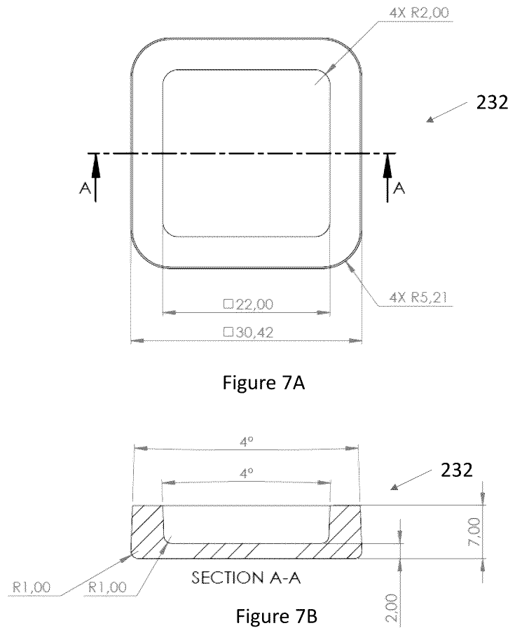

FIG. 7A and FIG. 7B illustrate another exemplary embodiment of the second layer 232.

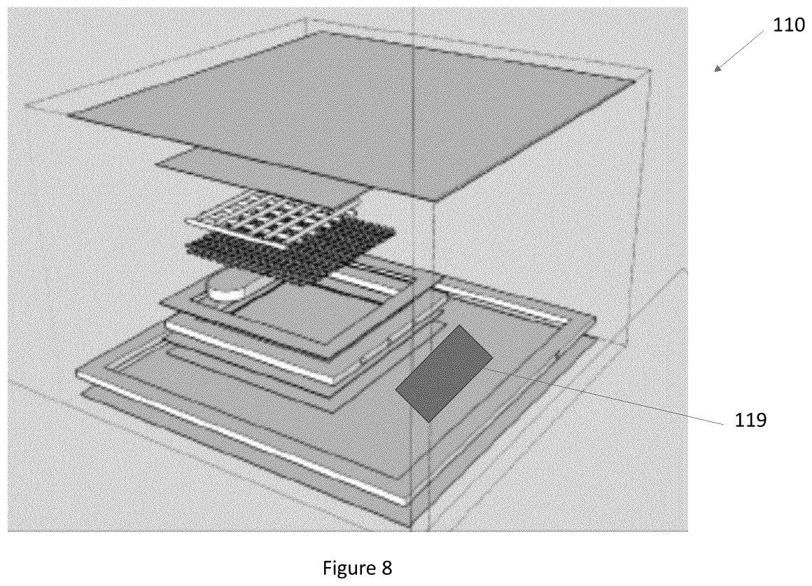

FIG. 8 illustrates the device 110 with the debridement aid 119 integrated. Any type, kind, number, and/or location of the debridement aid 119 may be utilized. For example, the debridement aid 119 may be provided within, or integrated with, the subsystem 10, and/or exterior to the device 110, 210, 610. Activation of the debridement aid 119 may be configured to provide mechanical agitation (e.g., vibration, such as by ultrasonics) the first portion 112 in exemplary embodiments, without limitation.

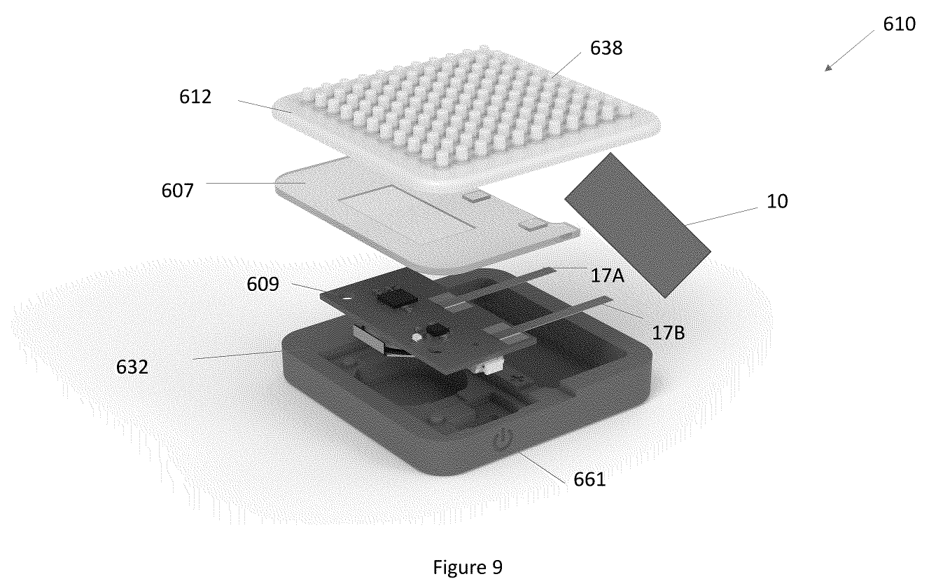

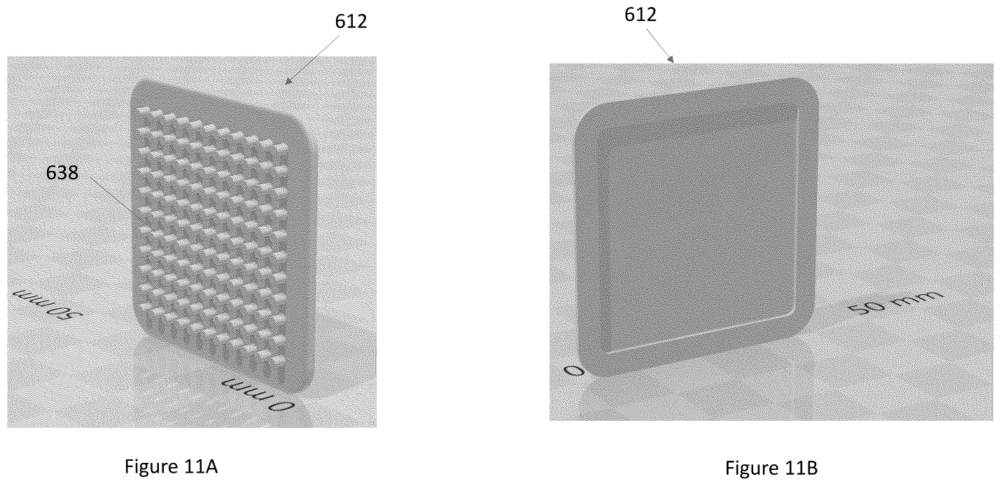

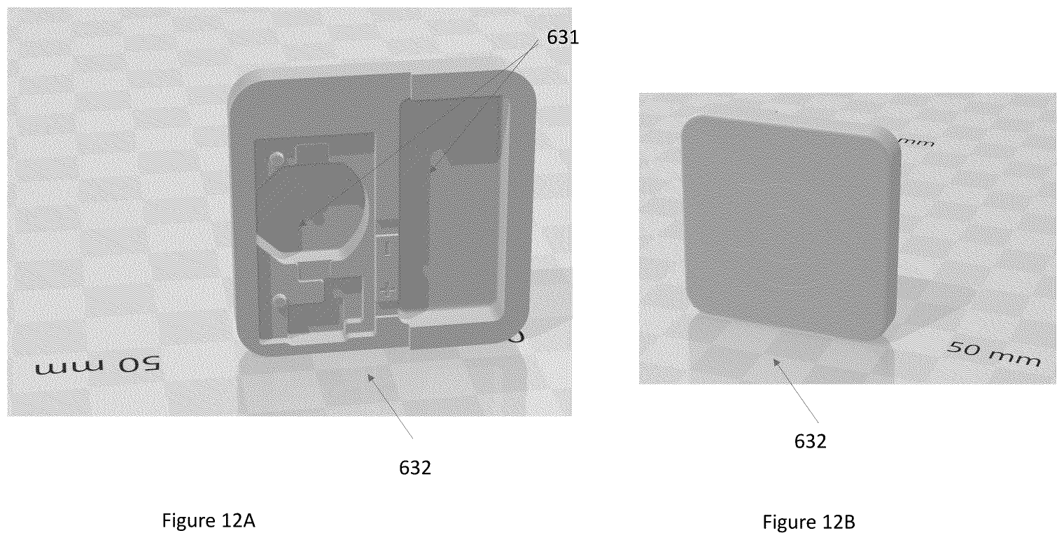

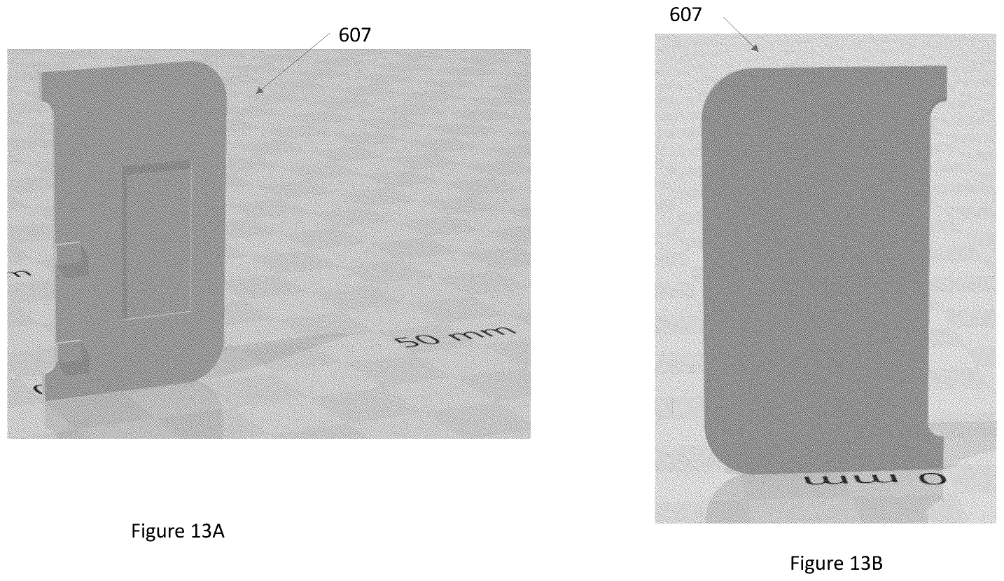

FIG. 9 through FIG. 13B illustrate another exemplary wound care device 610 with the oxygen producing subsystem 10. Similarly numbered components of the device 610 may be the same or similar to components of the device 110 and/or 210, though such is not necessarily the case (e.g., 112, 212 to 612). A first layer or portion 612 may comprise a number of protrusions 638. The protrusions 638 may be of the type or kind shown and/or described herein, such as being at least 1 mm in diameter, though any size and/or shape may be utilized. The first portion 612 may comprise pores, such as micro-pores, though such is not necessarily required.

The first portion 612 may be permanently or temporarily affixed to a second layer or portion 632 to enclose a controller 609. A cover layer or portion 607 may be provided between the controller 609 and the first portion 612, such as to provide a liquid barrier between the sensitive electronics of the controller 609 and the subsystem 10. Portions of the second portion 632 may be molded and/or comprise one or more cavities 631, such as to accommodate certain components or portions of the controller 609 and/or the subsystem 10. The cover 607 may be sized to accommodate the subsystem 10 in exemplary embodiments.

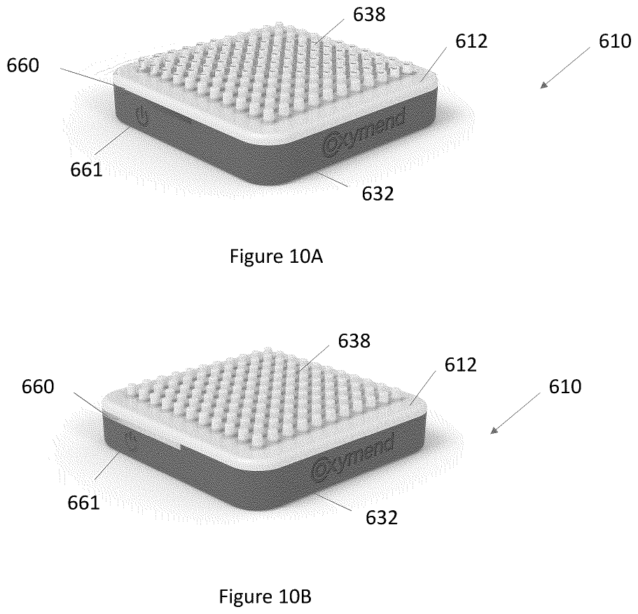

A portion, or all, of a user input device 616 may be located along an outer perimeter of the second portion 632, though any location may be utilized. A portion, or all, of an indicator 661 may be located along an outer perimeter of the second portion 632, though any location may be utilized. The indicator 660 may be illuminated (e.g., FIG. 10B) or not illuminated (e.g., FIG. 10A) to indicate that the device 610 and/or subsystem 10 are operational or not operational, respectively. In exemplary embodiments, control of the indicator 660 may be made by the controller 609 at least partially, if not entirely, in response to actuation of the user input device 616.

The controller 609, in exemplary embodiments without limitation, may comprise some or all of the electronic components shown and/or described herein. Such components may comprise, for example without limitation, printed circuit boards, microcircuits, processors, batteries or other power supplies, electrodes 17 (e.g., anode and/or cathode), buttons, switches, resistors, vibration or debridement devices or aids, motors, power modules, lights, capacitors, diodes, transistors, inductors, relays, integrated circuits, combinations thereof, or the like. Alternatively, or additionally, the device 610 may be configured to generate at least some power by way of piezo electricity, components for which may be provided within the device 610 and/or subsystem 10 and may be electronically controlled by the controller.

In exemplary embodiments, without limitation, the first portion 612 may be full or substantially gas permeable. For example, without limitation, the first portion 612 may be configured to provide a relatively high level of gas absorption and diffusion. The second portion 632 may be fully or substantially liquid impermeable and/or gas impermeable. For example, without limitation, the second portion 632 may be configured to be fully or substantially liquid tight to contain electrolytes or other materials. Alternatively, or additionally, the second portion 632 may be configured to provide a relatively low level of oxygen absorption and/or diffusion. This may encourage or force generated oxygen into the first portion 612, such as for transport into the wound.

The controller 609 may comprise a printed circuit board (PCB), which may be at least partially stored within a cavity, with various components located thereon, such as microcircuits, batteries, buttons, sensors (oxygen levels, movement of the device 610, temperature, pressure, combinations thereof, or the like), anodes 17A and/or cathodes 17B, data and/or network connectivity devices (e.g., USB ports, ethernet ports, near field communication device, wireless communication devices, radio transmitters/receivers, combinations thereof, or the like), combinations thereof, or the like. In exemplary embodiments, the anodes and cathodes 17 or other electrically conductive component(s) may extend form the PCB where they are electrically connected to the battery or other power source and into the electrolytes or other materials for producing oxygen, which may be stored (at least partially) in an adjacent cavity.

A debridement device 10 may optionally be included which is mounted to the PCB or provided separately.

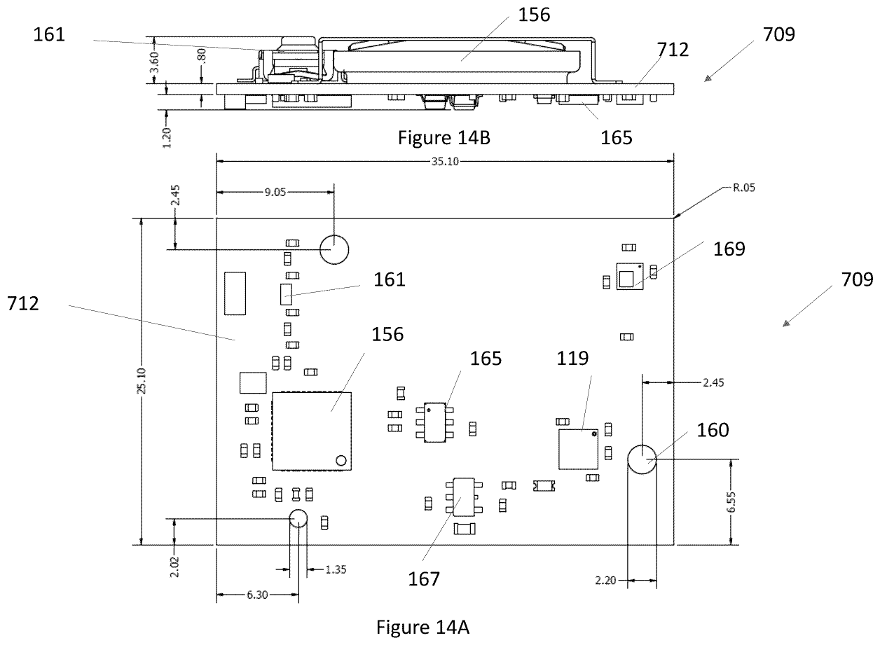

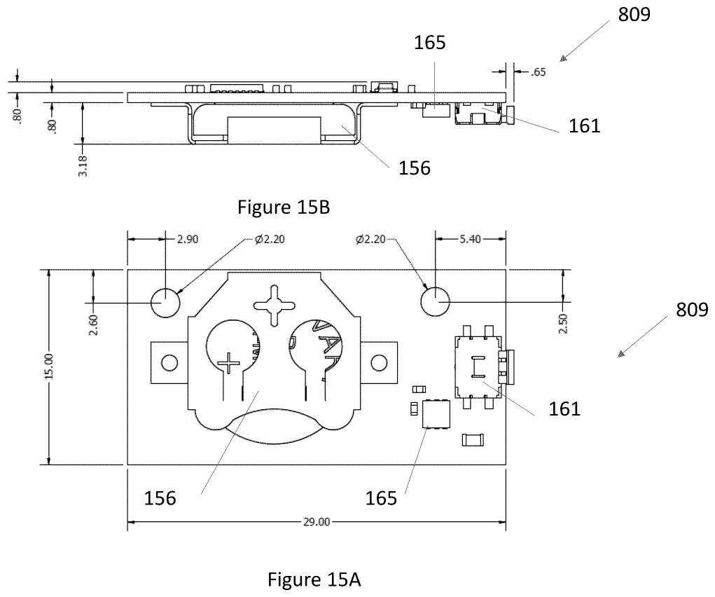

FIG. 14A through FIG. 15B illustrate exemplary embodiments of the controller 709, 809, respectively, either or both (e.g., in some combination thereof) may server as the controller 509, 609 or other controller shown and/or described herein. Similarly numbered components of the controllers 709, 809 may be the same or similar to components of the controller 509, though such is not necessarily the case (e.g., 509 to 709, 809). The controller 709 may comprise the sensors 169, the debridement aids 119, and/or related components, which are not necessarily required by the controller 809. However, any type or kind of controller 509, 708, 809, having any type, kind, number, and/or arrangement of electronic components may be utilized, such as to provide the components, features, and/or perform the steps shown and/or described herein.

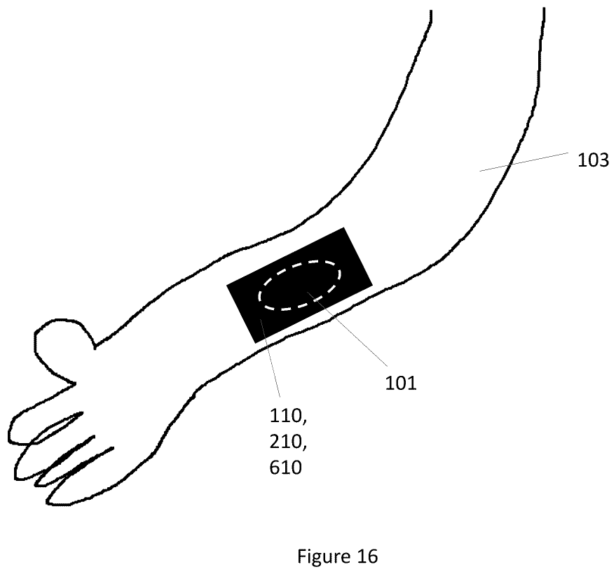

FIG. 16 is a plan view of the wound care device 110, 210, 610 provided at an exemplary wound 101. While the device 110, 210, 610 is illustrated as located at a wound 101 at an arm 103 of a user, the device 110, 210, 610 may be provided at any body part to cover some or all of any type or kind of wound, such as but not limited to, a laceration, abrasion, skin tear, puncture wound, surgical wound or incision, thermal, chemical, or electrical burn, bite, sting, gunshot or other projectile wound, contusion, blister, seroma, hematoma, crush injuries, ulcers, combinations thereof, or the like. The device 110, 210, 610 may be used with humans or other animals. The device 110, 210, 610 may be provided or secured to any body part or area, such as but not limited to by application of pressure, adhesive, bandage, wrapping, straps, combinations thereof, or the like.

The device 110, 210, 610 and/or subsystem 10 may be integrated with traditional bandages or other wound coverings. In other exemplary embodiments, without limitation, the subsystem 10 may be integrated with, or provided within, an otherwise fully or partially sealed environment, such as a wound covering, cast, bandage, brace, wound vacuum, device, and/or hyperbaric chamber, to promote healing. The device 110, 210, 610 and/or subsystem 10 may be used for various forms of wound care or other applications, such as but not limited to, respiratory assistance (e.g., metabolism, oxygen therapy, emergency survival oxygen, carbon monoxide/dioxide poison treatment), fire assistance, food production, odor removal, oxygen production for industrial applications (e.g., oxygen furnace, welding, wastewater treatment, metal cutting, bacterial killing), oxygen production for combustion applications (e.g., automotive engines, sanitary applications, rocket fuel), combinations thereof, or the like.

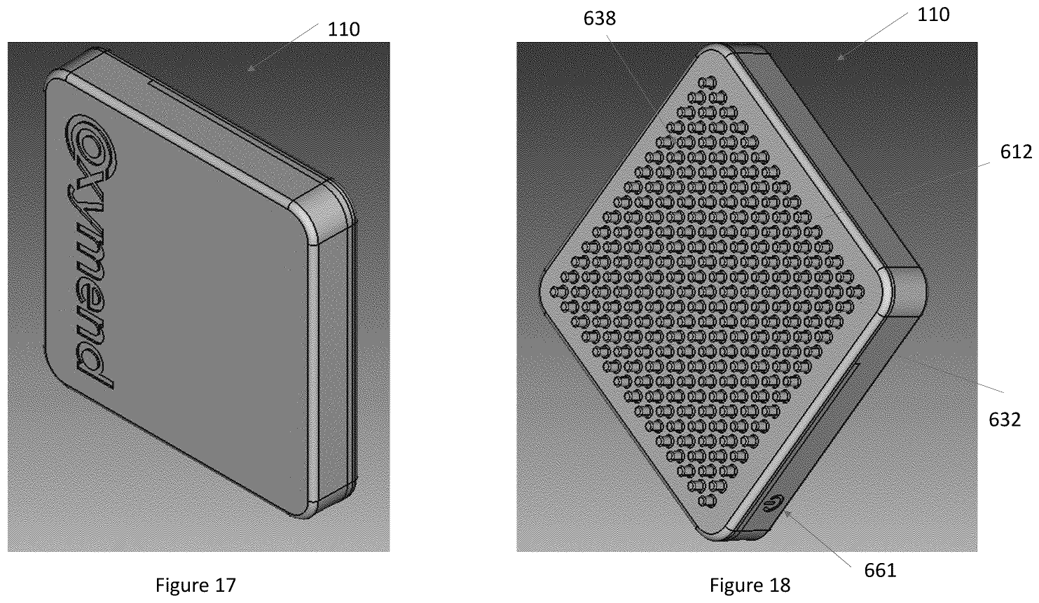

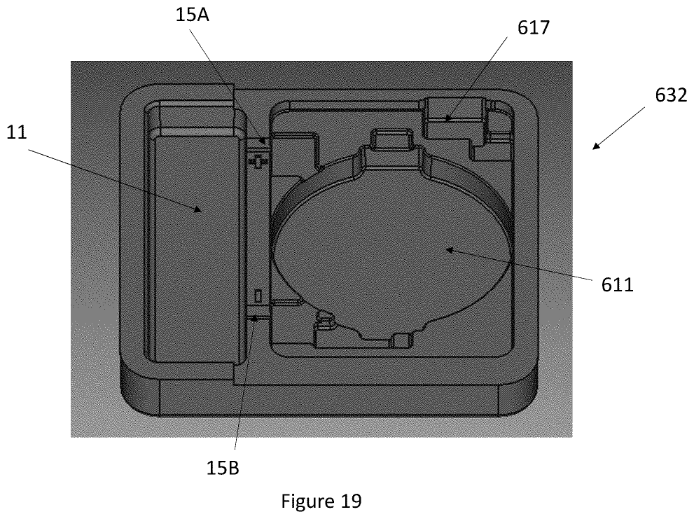

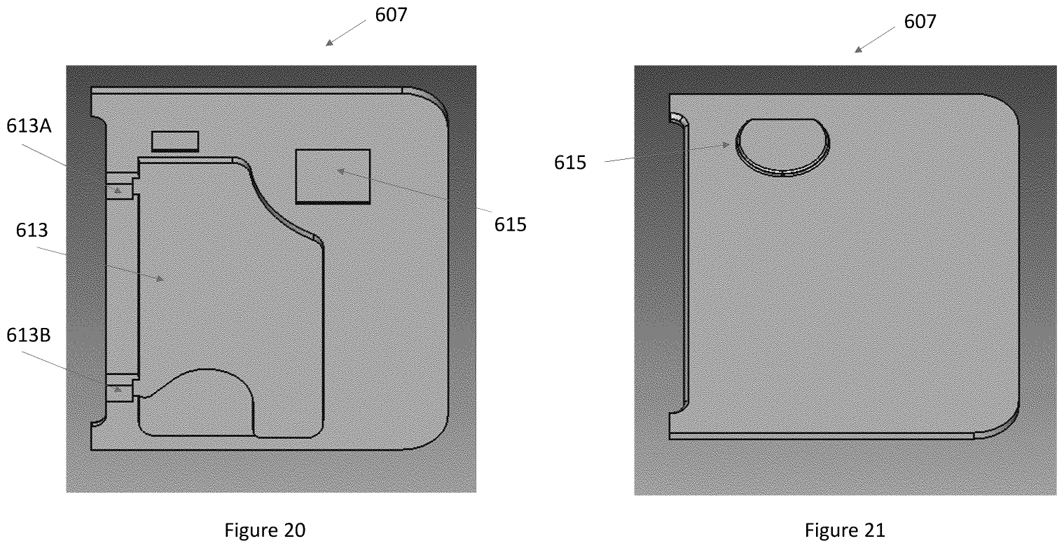

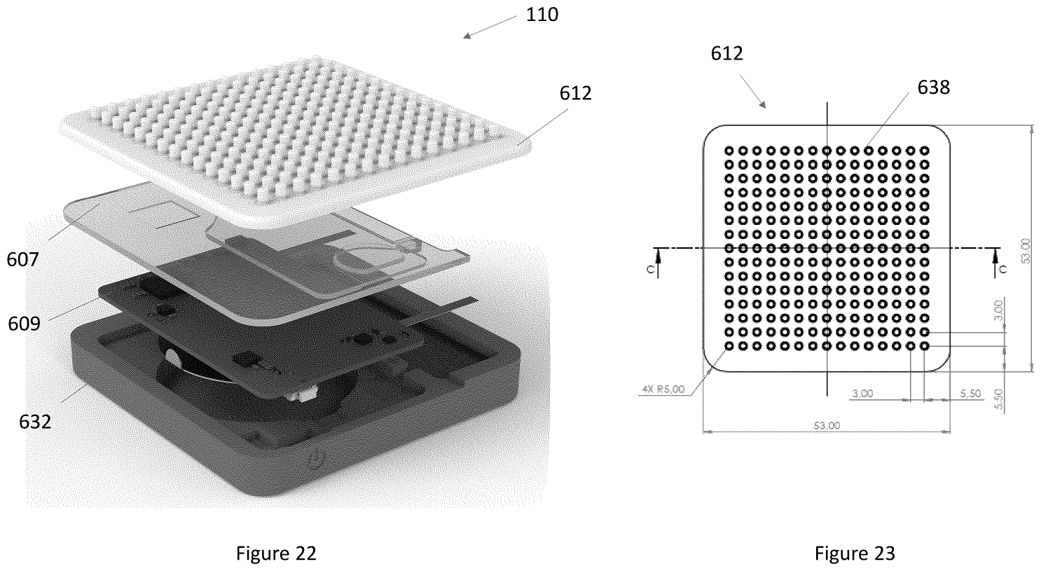

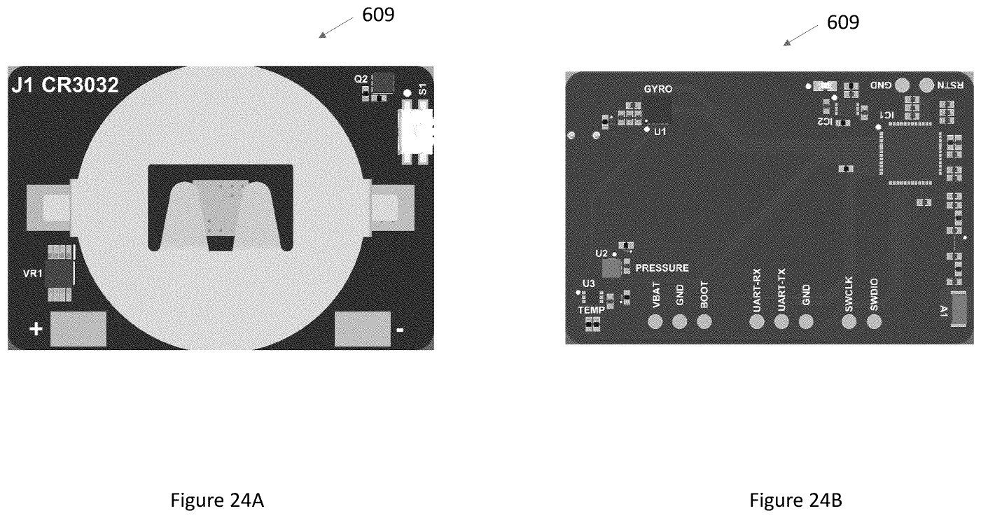

FIG. 17 through FIG. 24B illustrate another exemplary embodiment of the device 110. A number of protrusions 638, such as of the type and/or kind discussed herein, may extend from a user contact surface 612. The protrusions 638 may be at least 1 mm in diameter, though any size and/or shape may be utilized.

An indicator and/or actuatable area 661 may be provided at a housing 632. The area 661 may comprise touch sensitive materials and/or be deformable to permit actuation of a button below. The user contact surface 612 may be configured to mate with the housing 632 to define an entirely or substantially closed area, which may be fluidly sealed but gas permeable or resistant in exemplary embodiments, without limitation.

The housing 632 may be generally square or rectangular in shape though any size and/or shape may be utilized. The housing 632 may comprise one or more cavities configured to accommodate various components of the device 110 or subsystem 10. For example, without limitation, the housing 632 may comprise a subsystem cavity 11. The subsystem cavity 11 may be configured, such as by size and/or location, to accommodate some or all of the subsystem 10 including but not limited to electrolytes and/or electrolysis components thereof. The subsystem cavity 11 may be substantially cuboid in shape, though any size or shape may be utilized. Alternatively, or additionally, the housing 632 may comprise a controller cavity 611. The controller cavity 611 may be configured, such as by size and/or location, to accommodate some or all of the controller 609, such as but not limited to a battery portion, electrode, resistor, combinations thereof, or the like. The controller cavity 611 may be substantially cylindrical in shape, though any size or shape may be utilized.

The housing 632 may comprise one or more channels 15. The channels 15 may be configured to accommodate components of the subsystem 10, controller 609, or the like. The channels 15 may extend between the controller cavity 611 and the subsystem cavity 11. For example, without limitation, first and second channels 15A, 15B may be provided to accommodate first and second electrodes 17A, 17B (e.g., anode and cathode, or other electrically conductive conduit or component) which may extend from the controller 609, such as a battery, other power source, and/or resistor portion thereof, into to the subsystem cavity 11, such as into the electrolytes or other components thereof. This may facilitate electrical connection between the battery or other power sources and the subsystem 10 for generating oxygen, such as through an electrolysis process.

The device 110 may comprise an internal cover 607. The internal cover 607 may be interposed between a portion of the housing 632, such as between an upper portion or surface(s) thereof, and a portion of the user contact surface 612, such as lower portion or surface(s) thereof. The internal cover 607 may define, at least in part, the subsystem cavity 11 and/or the controller cavity 611, though such is not required. In exemplary embodiments, without limitation, the subsystem cavity 11 and/or the controller cavity 611 are fully or substantially enclosed, such as but not limited to in a fluid tight but gas permeable fashion, such as within the larger enclosure of the device 110 defined by the housing 632 and the user contact portion 612. In this fashion, one or more internal enclosures may be provided within the larger enclosure of the device.

The internal cover 607 may comprise one or more indentation, protrusions, combinations thereof, or the like. The indentation, protrusions, combinations thereof, or the like may be configured to accommodate and/or secure certain components. In exemplary embodiments, without limitation, the internal cover 607 may comprise a controller indentation 613, which may be configured to accommodate some or all of the controller 606. The internal cover 607 may comprise one or more channels 613, which may extend from the controller indentation 613 to an edge of the internal cover 607 and/or a space at or adjacent the subsystem cavity 11 when the device 110 is assembled. The channels 613 may comprise a first channel 613A and/or a second channel 613B configured to accommodate the electrodes 17A, 17B, respectively.

In exemplary embodiments, without limitation, the internal cover 607 may comprise, or may serve as, an electrical insulator. The internal cover 607 may, alternatively or additionally, provided separation between the subsystem cavity 11 and the controller cavity 611. The subsystem cavity 11 may be enclosed, at least in part, by the user contact portion 612, though such is not required.

The subsystem cavity 11 may accommodate one or more of an electrode mesh, separator, electrolyte solution, portions of the electrodes, combinations thereof, or the like, which may reside at least partially therein and be operated to selective generate oxygen.

The controller 609 and components thereof may form a control subsystem for the device 110.

Any dimensions shown and/or described herein are merely exemplary and are not intended to be limiting.

One of more antimicrobial gels may be provided at the device 110. In exemplary embodiments, without limitation, such antimicrobial gels are provided as a coating on at least the user contact portion 612. Such coatings may be provided at manufacture, before placement at a patient, combinations thereof, or the like.

One of more oxygen delivery enhancement gels may be provided at the device 110. These may include, for example without limitation haemoglobin-based materials, hyaluronic acid, combinations thereof, or the like. In exemplary embodiments, without limitation, such oxygen delivery enhancement gels are provided as a coating on at least the user contact portion 612. Such coatings may be provided at manufacture, before placement at a patient, combinations thereof, or the like.

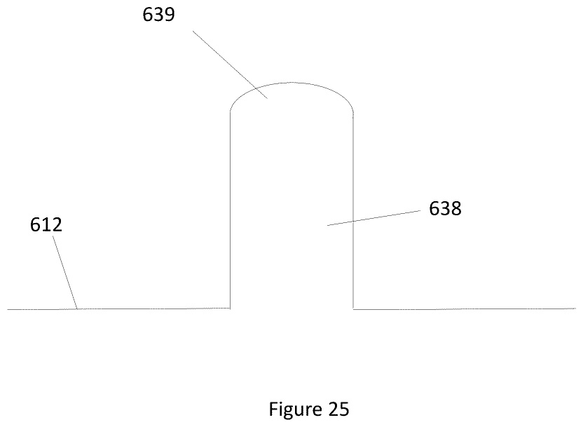

FIG. 25 is a detailed side view of an exemplary projection 638, any number and arrangement of which may be provided the user contact portion 612. The projection 638 may comprise rounded upper portion 639. In other exemplary embodiments, the top portion may comprise a bulbous or otherwise varying shape section which is provided at distal end of the otherwise generally columnar, conical, and/or pyramidal projection 638. This rounded upper portion 639 and/or varied shape may increase oxygen diffusion, wound penetration, wound interface, combinations thereof, or the like. Any size, shape, and/or type of upper portion 639 may be used in conjunction with any size, shape, and/or type of projection 638. The projections 638 of any device 610 may be of a generally same or different size, shape, type, or the like. Any number and type of projections 63 may be provided in any arrangement at the user contact portions 612.

Any embodiment of the present invention may include any of the features of the other embodiments of the present invention. The exemplary embodiments herein disclosed are not intended to be exhaustive or to unnecessarily limit the scope of the invention. The exemplary embodiments were chosen and described in order to explain the principles of the present invention so that others skilled in the art may practice the invention. Having shown and described exemplary embodiments of the present invention, those skilled in the art will realize that many variations and modifications may be made to the described invention. Many of those variations and modifications will provide the same result and fall within the spirit of the claimed invention. It is the intention, therefore, to limit the invention only as indicated by the scope of the claims.

Certain operations described herein may be performed by one or more electronic devices. Each electronic device may comprise one or more processors, electronic storage devices, executable software instructions, combinations thereof, and the like configured to perform the operations described herein. The electronic devices may be general purpose computers or specialized computing devices. The electronic devices may comprise personal computers, smartphone, tablets, databases, servers, or the like. The electronic connections and transmissions described herein may be accomplished by wired or wireless means. The computerized hardware, software, components, systems, steps, methods, and/or processes described herein may serve to improve the speed of the computerized hardware, software, systems, steps, methods, and/or processes described herein.

### Appendix

Further description is provided in this appendix of exemplary actual and/or hypothetical experiments and results for the subsystem 10 and/or device 110, 210, 610 shown and/or described herein. These experiments and the results are provided as mere examples and are not intended to be limiting. Other results may be achieved using certain embodiments of the subsystem 10 and/or device 110, 210, 610 shown and/or described herein.

### Experimental Series 1

A hole was drilled into a battery type Ni-electrode and a stainless-steel wire was inserted as current collector. A stainless-steel mesh was cut into a counter electrode (hydrogen and oxygen electrode). The size of the counter electrode was about 3× the size of the Ni electrode. The counter electrode is without catalyst. Large size is chosen to prevent it from being diffusion limiting for the experiment. The electrodes were placed in a plastic cup with Styrofoam lid and an KOH electrolyte was used (1 g KOH pellets mixed in 100 ml distilled water).

One Ni-electrode (one side surface area of 2 cm2) was used and connected for O2 evolution. Current was set at 0.05 A or 50 mA and voltage was recorded in the following table:

| | |
|---|---|
| Time | Voltage/V |

| | | |
|---|---|---|
| 1:30 | min | 0.7 |
| 5 | min | 0.8 |
| 10 | min | 0.8 |
| 20 | min | 1.0 |
| 33 | min | 1.0 |
| 50:30 | min | 1.4 |
| 52 | min | 2.0 |
| 53 | min | Stopped |

The electrode connections were then switched for H2 evolution. For the Ni-electrode this means that NiOOH converts to Ni(OH)2. Current was set at 50 mA, time and voltage recorded in the following table:

| | |
|---|---|
| Time | Voltage/V |

| | | |
|---|---|---|
| 1 | min | 2.1 |
| 10 | Min | 2.1 |
| 40 | Min | 2.1 |
| 41 | min | 2.2 |
| 52 | min | 2.2 |

| | |
|---|---|
| 1 hour | 2.2 |
| 5 min | |
| 1 hour | 2.2 |
| 20 min | |
| 1 hour | 2.3 |
| 22 min | |
| 1 hour | 2.3 |
| 32 min | |

| | | |
|---|---|---|
| 2 | hours | 2.2 |
| 3 | hours | 2.1 |
| 3 | hours | Stopped |

The electrode connection was then switched to O2 evolution. The test was now done at constant voltage. The voltage was set at 0.4 V and current and time monitored. Results are given in the following table:

| Time | Current/mA |
|---|---|
| 1 min | 70 |
| 45 min | 40 |
| 1 hour 23 min | 0 |

The electrode connections were then switched back to H2 evolution. Now with constant voltage set at 2V.

| | |
|---|---|
| Time | Current/mA |

| | | |
|---|---|---|
| 1 | min | 10 |
| 9 | min | 30 |
| 23 | min | 40 |
| 1 | hour | 40 |

| | |
|---|---|
| 1 hour | 10 |
| 8 min | |
| 1 hour | 40 |
| 45 min | |
| 2 hours | 30 |
| 3 min | |
| 2 hours | 30 |
| 35 min | |

| | | |
|---|---|---|
| 3 | hours | 30 |
| 3 | hours | Stopped |

The observed drop in current at 1 hour 8 min was due to loss of electrical contact.

The electrode connections were then switched back to O2 evolution. Constant voltage was set at 0.4 V. Care was taken to make sure contact was better attached. Results given in table below:

| | |
|---|---|
| Time | Current/mA |

| | | |
|---|---|---|
| 1 | min | 60 |
| 2 | min | 70 |
| 5 | min | 90 |
| 15 | min | 90 |
| 30 | min | 80 |
| 1 | hour | 60 |

| | |
|---|---|
| 1 hour | 30 |
| 8 min | |
| 1 hour | 30 |
| 10 min | |
| 1 hour | 20 |
| 20 min | |
| 1 hour | 10 |
| 30 min | |
| 1 hour | Stopped |
| 33 min | |

### Experimental Series 2

A Ni-electrode and stainless-steel electrode was then placed in a PTFE bag filled with 1 M KOH electrolyte. The bag was not sealed on the top. A plastic mesh was used as a divider to prevent short circuit between the two electrodes. Voltage was set at 0.8-0.9 V from an external power source. 200 mA O2 formation was recorded.

### Experimental Series 3

3-D printed holders for the electrodes were prepared. Two electrode holders for the stainless-steel and one for the Ni-electrode. The concept was to place one stainless-steel electrode on each side of the Ni-electrode to utilize both sides of the Ni-electrode.

The Ni-electrode holder was sized to fit 5 battery Ni-electrodes. This size was determined based on the following input. 2.8 A was calculated to be current equivalent to 10 L/min O2 production.

For the first test, the electrodes were connected for)2 evolution. A short test was run with voltage set at 0.4 V as shown in the table below.

| Time | Current/mA |
|---|---|
| 1 min | 300 |
| 4 min | 300 |

A sweep was then performed in both directions (from low to high and high to low voltage).

| | Current/mA low | Current/mA high |
|---|---|---|
| Voltage/V | to high sweep | to low sweep |
| 0.1 | 0 | 0 |
| 0.2 | 10 | 30 |
| 0.3 | 110 | 170 |
| 0.4 | 260 | 290 |
| 0.5 | 380 | 390 |
| 0.6 | 480 | 510 |
| 0.7 | 620 | 670 |
| 0.8 | 750 | 750 |

Capacity of the Ni-electrode taken from the battery is in the 100 mAh range for O2 evolution. We observed some gas evolution from the stainless-steel wire that makes capacity measurement difficult. Battery company reports a 150 mAh capacity. This is probably achievable at lower currents.

The weight of the Ni-electrode is 1.35 g giving a capacity of 70 mAh/g (measured) vs. 110 mAh/g (reported). Reported literature capacity is 210 mAh/g. The small electrodes and high amount of current collector used probably results in the reduction of capacity vs. the literature data.

Voltage for hydrogen evolution is higher than expected for 50 mA current. This is probably related to the stainless-steel electrode and should be easily fixed with a NiPx catalyst.

Voltage for the O2 evolution is in the target range. Improvements with increased surface area is probably easiest as observed good O2 evolution rates at 0.2-0.3 V.

If we activate the electrode with NiPx we should test if we need a separate O2 and H2 electrode or if we can run both on the same electrodes. Test to understand if O2 reaction damage NiPx catalyst or if NiPx catalyst reduces O2 evolution rate.

What is claimed is:

1. A device for supplying oxygen to a patient for treatment of a wound or condition,
said device comprising:
an outer housing comprising a user contact surface comprising protrusions; and
an oxygen generating subsystem located inside the outer housing and configured to electrochemically generate oxygen, wherein at least the user contact surface and the protrusions are oxygen permeable.

2. The device of claim 1 wherein:
the outer housing is liquid impermeable.

3. The device of claim 2 wherein:
the oxygen generating subsystem is located within an internal housing which is gas permeable for at least oxygen and liquid impermeable.

4. The device of claim 3 wherein:
the protrusions comprise finger-like projections spaced apart at the user contact surface; and
the protrusions are generally conical in shape and at least 1 mm in diameter.

5. The device of claim 4 wherein:
the protrusions comprise silicone;
the user contact surface comprises silicone; and
portions of the housing other than the user contact surface comprise one or more materials with relatively low gas permeability and absorption.

6. The device of claim 1 wherein:
the oxygen generating subsystem comprises: a controller, a power source, an electrolyte reservoir, an anode, and a cathode.

7. The device of claim 6 wherein:
the outer housing comprises a first cavity for the controller, a second cavity for the electrolyte reservoir, a first channel for the anode to extend from the power source to the electrolyte reservoir, and a second channel for the cathode to extend from the power source to the electrolyte reservoir.

8. The device of claim 6 wherein:
said oxygen generating subsystem comprises NiOOH and is configured to periodically generate hydrogen instead of oxygen.

9. The device of claim 8 wherein:
said oxygen generating subsystem is configured to generate between about 0.01 to about 50 ml oxygen/hr under sea level ambient air pressure and air temperatures between about 32° F. and 100° F.

10. The device of claim 1 further comprising:
a debridement device located within the outer housing and configured to mechanically agitate the user contact surface and protrusions.

11. The device of claim 1 further comprising:
one or more sensors located within the outer housing and in electronic communication with the controller; and
a network communication device in electronic communication with the controller, wherein the controller is configured to receive readings from the one or more sensors and wirelessly transmit the readings to at least one remote electronic device by way of the network communication device.

12. The device of claim 11 wherein:
the one or more sensors comprise at least one of: a temperature sensor, a pressure sensor, an oxygen sensor, and a movement sensor.

13. A method for supplying oxygen to a patient for treatment of a wound or condition,
said method comprising:
placing a device at a wound of the patient such that protrusions extending from a user contact surface of an outer housing for the device extend through one or more biofilms of the wound;
supplying power from an internal power source of the device, by way of one or more electronic commands issued from a controller of the device, to an oxygen generating subsystem located inside the outer housing to electrochemically generate oxygen within the device; and
allowing the generated oxygen to diffuse through the user contact surface and the protrusions, which are gas permeable at least as to oxygen, and into the wound below the one or more biofilms.

14. The method of claim 13 wherein:
the protrusions comprise finger-like projections of substantially conical shape having a minimum diameter of at least 1 mm; and
the protrusion and the user contact surface comprise silicone.

15. The method of claim 14 wherein:
said device comprises an internal cover and a housing portion;
said housing portion comprises a first cavity configured to accommodate at least a portion of a printed circuit board comprising the controller and a power supply for the device, a second cavity configured to accommodate an electrolyte reservoir of the oxygen generation subsystem, a first channel for an anode extending from the printed circuit board into the electrolyte reservoir, and a second channel for a cathode extending from the printed circuit board into the electrolyte reservoir; and
the anode and the cathode are electrically connected to the power supply.

16. The method of claim 15 further comprising:
monitoring, by way of sensors installed at the printed circuit board and the controller, oxygen produced by the oxygen generation subsystem; and
periodically operating, by way of commands issued from the controller, the oxygen generation subsystem in a hydrogen producing mode.

17. The method of claim 16 wherein:
operating said oxygen generating subsystem, by way of the controller, to generate between about 0.01 to about 50 ml oxygen/hr under sea level ambient air pressure and ambient air temperatures between about 32° F. and 100° F.

18. The method of claim 17 further comprising:
receiving, at the controller from one or more sensors of the device, oxygen readings and movement readings; and
electronically transmitting, from the controller to one or more remote devices by way of a network communication device at the device, the oxygen readings and movement readings.

19. The method of claim 18 further comprising:
periodically activating, by way of one or more electronic commands issued from a controller of the device, a debridement device to mechanically agitate the wound by induced movement of the protrusions.

20. A device for supplying oxygen to a patient for treatment of a wound or condition,
said device comprising:
an outer housing comprising a first portion and a user contact surface which are joined to form a substantially liquid-tight enclosure;
finger-like, conical shaped protrusions having a minimum diameter of at least 1 mm extending from the user contact surface, where said user contact surface and said protrusions are gas-permeable and comprise silicone, and where the first portion is relatively less gas permeable;
a printed circuit board (“PCB”) located within the outer housing;
a controller mounted to said PCB;
a battery mounted to said PCB electrically connected to said controller;
a debridement device mounted to said PCB, electrically connected to said battery, in electronic communication with said controller, and configured to emit ultrasonic waves toward the user contact surface when activated;
an anode mounted to said PCB and electrically connected to said battery;
a cathode mounted to said PCB and electrically connected to said battery;
sensors mounted to said PCB, electrically connected to said battery, and in electronic communication with said controller, said sensors comprising at least an accelerometer and an oxygen sensor;
a wireless communication device mounted to said PCB and in electronic communication with the controller;
an indicator light mounted to said PCB, electrically connected to said battery, and in electronic communication with said controller;
a first cavity located in said first portion of said outer housing configured to accommodate at least part of said PCB and said battery;
a second cavity located in said first portion of said outer housing;
a reservoir of electrolyte material located in the second cavity;
an internal cover located within the outer housing which secures and accommodates at least part of said PCB;
a first channel extending between said first cavity and said second cavity, where said first channel is defined, at least in part, by said first portion of said outer housing and said internal cover, and where said anode extends through said first channel; and
a second channel spaced apart from said first channel and extending between said first cavity and said second cavity, where said second channel is defined, at least in party, by said first portion of said outer housing and said internal cover, and where said cathode extends through said second channel;
wherein said controller comprises one or more processors and one or more electronic storage devices comprising software instructions, which when executed, configure the one or more processors to:
apply power from the battery to the electrolyte reservoir to generate oxygen;
monitor data from said oxygen sensor to determine oxygen production levels;
activate certain of said indicator light when said oxygen sensor indicates that oxygen is detected an amount above a predetermined threshold is present;
adjust power supplied form the battery based on the oxygen production levels;
periodically apply power form the battery to the ultrasonic debridement device to cause emission of ultrasonic waves towards the user contact surface;
monitor data from said accelerometer to determine movement of the device;
generate and transmit an electronic notification to at least one remote electronic device when the movement of the device is above a predetermined threshold; and
transmit data regarding the oxygen production levels to at least one other remote electronic device.
